(19) United States
(12) Patent Application Publication
(10) Pub. No.: US 2003/0134293 A1
(43) Pub. Date: Jul. 17, 2003

(54) Method for rapid and accurate identification of microorganisms

(76) Inventor: Zhiping Liu, Mountain View, CA (US)

Correspondence Address: Shantanu Basu, Morrison & Foerster LLP, 755 Page Mill Road, Palo Alto, CA, 94304-1018, US

(21) Appl. No.: 10/151,484

(22) Filed: May 16, 2002

Related U.S. Application Data
(63) Continuation-in-part of application No. PCT/US00/31579, filed on Nov. 16, 2000.
(60) Provisional application No. 60/165,881, filed on Nov. 16, 1999.

(51) Int. Cl. C12Q 1/70; C12Q 1/68; C12P 19/34

(52) U.S. Cl. 435/6; 435/5; 435/91.2

(57) ABSTRACT

Methods and compositions useful for rapid identification of microorganisms are provided. Methods and compositions useful for rapid and simultaneous identification in a biological sample of multiple microorganisms, including bacteria, yeast, fungi and viruses are also provided. The methods and compositions utilize amplification techniques and sequence specific hybridization to detect species specific polynucleotide sequence in a sample. Novel methods for coupling oligonucleotide probes to glass surfaces with increased efficiency are also provided.

### CROSS-REFERENCE TO RELATED APPLICATIONS

This application is a continuation-in-part of International Application PCT/US00/31579, with an international filing date of Nov. 16, 2000, published in English under PCT Article 21(2) (International Publication Number WO 01/36683 A2, published May 25, 2001), which claims priority under 35 U.S.C. §119 to U.S. Provisional Application Serial No. 60/165,881, filed Nov. 16, 1999, and U.S. application Ser. No. 09/479,457 filed Jan. 6, 2000, the disclosures of which are incorporated herein by reference.

### BACKGROUND OF THE INVENTION

Infectious diseases represent an increasingly serious public health concern. Since multiple infectious agents can cause the same or similar symptoms, the identification of the pathogen is crucial for the correct diagnosis and proper treatment of the illness. The etiologic agents for pneumonia and meningitis, to name just two serious diseases, include more than a dozen different bacteria and several viruses and fungi. Most of the current diagnostic procedures involve culturing the bacteria for identification, a process that usually requires several days and often gives negative results. Culturing is not only a lengthy process, but certain pathogens (i.e., mycoplasma, mycobacteria, and viruses) are notoriously difficult to grow outside the host. Current “non-culture” techniques for detecting and identifying a pathogen are designed for a specific pathogen, even though many different pathogens can cause the same symptoms and many patients have mixed infections. To obtain a diagnosis, the physician is forced to use several assays for a single patient, which is a very expensive undertaking. To illustrate the scope of the problem, consider the common etiologic agents for pneumonia, which includes: the classic pathogens Streptococcus pneumoniae, Enterobacteriaceae, Staphylococcus aureus, Chlamydia pneumoniae, Escherichia coli, Legionella pneumophila, and Pseudomonas aeruginosa; the atypical agents Mycoplasma pneumonia, Mycobacteria, and Pneumocystis carinii (predominantly in immuno-compromised patients); and a variety of viruses and fungi (Kayser 1992; Tan, 1999). For bacterial meningitis, major etiologic agents include: Neisseria meningitidis, Haemophilus influenza, and Streptococcus pneumoniae (Tunkel and Scheld, 1993). Since the proper medical treatment for these infections varies substantially depending on the agent, it is important to rapidly and accurately identify the pathogen.

There is currently a desire for a faster and more cost effective method for determining the identity of various pathogens. Such methods are beneficial in that they can more readily determine the proper therapeutic treatments and determine the best method of resolving a microorganism-associated contamination or infection.

### SUMMARY OF THE INVENTION

DNA hybridization probes and PCR offer considerable promise for the development of microbial diagnostics (Abele-Horn et al., 1998; Ramirez et al., 1996). The present invention takes advantage of the fact that certain coding sequences are highly conserved in a number of organisms (e.g., eubacteria). By properly choosing PCR primers from among these conserved sequences, one set of PCR primers (or a set of degenerate primers) can be used for the amplification of an unknown DNA sample (with several possible and different genomic origins) for the purpose of revealing its identity. To achieve further amplification, an additional set of primers can be designed based on the same principle for nested-PCR (i.e., a second set of primers within the bounds of the first set of primers). In conjunction, hybridization probes will be chosen from the less conserved sequences (horizontally in evolution) flanked by the PCR primers. The same principles can be applied for identifying any number of microorganisms including, for example, viruses and eukaryotic cells, such as fungi.

In one embodiment, the invention provides a method of identifying an organism among a population of organisms in a biological sample, the method comprising obtaining genetic material from the sample; contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and is characteristic of the organism; amplifying the target sequence; contacting a solid support comprising a probe substantially complementary to the target sequence with the amplified target sequence; and detecting hybridization of the target sequence to the probe, wherein hybridization is indicative of the presence of the organism in the sample.

In another embodiment, the invention provides a method of diagnosing a disease or disorder associated with an organism, comprising obtaining genetic material from a sample; contacting the genetic material with at least a first primer and at least a related second primer corresponding to a pair of conserved regions in the genome of a population of organisms, wherein the first primer hybridizes upstream and the second primer hybridizes downstream of a target sequence in the genetic material in the sample, and further wherein the target sequence is less conserved than the primer binding sequences and is characteristic of the organism; amplifying the target sequence; contacting a solid support comprising a probe substantially complementary to the target sequence with the amplified target sequence; and detecting hybridization of the target sequence to the probe, wherein hybridization is indicative of the presence of the organism in the sample and correlating the organism to the disease or disorder.

In yet another embodiment, the invention provides an array of oligonucleotide probes immobilized on a solid support, the array comprising a plurality of probes having a sequence corresponding to a species specific polynucleotide target sequence wherein the species specific target sequence is flanked by oligonucleotide sequence that are conserved across a population of organisms. The population of organisms can be of the same family or genus or cause the same disease or disorder.

In another embodiment, the invention provides a kit comprising, at least one container having therein an at least one oligonucleotide primer complementary to a conserved region of genetic material in a population of organisms; and a solid support having attached thereto a species-specific probe capable of hybridizing to a target sequence, the target sequence flanked by the at least one primer.

In one embodiment, the invention provides a method of identifying at least two organisms from a population of organisms in a biological sample, comprising obtaining genetic material from the biological sample; contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and each target sequence is characteristic of one of the at least two organisms; amplifying the target sequence; providing a solid support comprising at least two probes selected from the at least two different organisms, wherein the at least two probes comprise sequences that are substantially complementary to the target sequence in the organism from which the probe sequences were selected; contacting the solid support with amplification products of the amplified target sequence; and detecting hybridization of the target sequence to the probe, wherein hybridization to a probe is indicative of the presence of the corresponding organism in the sample.

In another embodiment, the invention provides a method of distinguishing a presence of at least two organisms from a population of organisms in a biological sample, comprising obtaining genetic material from the biological sample; contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and each target sequence is characteristic of one of the at least two organisms; amplifying the target sequence; providing a solid support comprising at least two probes selected from the at least two different organisms, wherein the at least two probes comprise sequences that are substantially complementary to the target sequence and differentially hybridize to the target sequence depending on a hybridization condition; contacting the solid support with amplification products of the amplified target sequence under a hybridization condition wherein hybridization to a probe corresponding to any one of the at least two organisms is preferred; and detecting hybridization of the target sequence to the probe corresponding to any one of the at least two organisms, wherein hybridization to the probe is indicative of the presence of the corresponding organism in the sample. In an embodiment, the at least two different organisms may be selected from two different organisms comprise bacteria, yeast, paramecia, trypanosoma, unicellular eukaryotes, and viruses.

In yet another embodiment, the invention provides a method of identifying a target sequence in a biological sample, comprising obtaining genetic material from the biological sample; contacting the genetic material with at least a first primer and at least a related second primer corresponding to a pair of conserved regions in the genome of a population of organisms, wherein the first primer hybridizes upstream and the second primer hybridizes downstream of a target sequence in the genetic material in the sample, and further wherein the target sequence is less conserved than the primer binding sequences; amplifying the target sequence; and determining the sequence of amplification products of the amplified target sequence. Furthermore, the invention provides a method for identifying an organism associated with the sequenced target sequence by comparing the sequence of the amplified target with a known sequence of the corresponding target in the organism.

In one aspect of the invention, a method is provided for increasing the efficiency of coupling of an oligonucleotide to a solid substrate, the method comprising applying a positive electrostatic potential to a surface of the solid substrate, whereby the positive electrostatic potential increases a concentration of oligonucleotides and negatively charged molecules to the surface of the solid substrate.

In another aspect of the invention, a method is provided for increasing the efficiency of coupling of an oligonucleotide to a glass substrate by forming an Epoxy derivative of a surface of the glass substrate, the method comprising applying an Epoxy derivative to the surface of the glass substrate.

### BRIEF DESCRIPTION OF THE DRAWINGS

FIG. 1 shows an alignment of conserved sequence used as primers in the methods and compositions of the invention.

FIG. 2 schematically illustrates a method of using a microorganism identification chip involving hybridization of PCR amplification products of an unknown sample using primers according to the present invention to specific probes immobilized on a solid substrate.

FIG. 3 shows the effect of primer concentration on amplification by individual set of PCR primers and mixed PCR primers for a RecA gene fragment.

FIG. 4 illustrates a comparison between specific (FIG. 4A) and mixed (FIG. 4B) primers.

FIG. 5 shows the results from a mutation that disrupts 3′-end hair-pin formation in a primer for S. aureus FtsY gene.

### DETAILED DESCRIPTION OF THE INVENTION

It must be noted that as used herein and in the appended claims, the singular forms “a,” “and,” and “the” include plural referents unless the context clearly dictates otherwise. Thus, for example, reference to “a primer” includes a plurality of such primers and reference to “the primer” includes reference to one or more primers and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the methodologies, which are described in the publications, which might be used in connection with the presently described invention. The publications discussed above and throughout the text are provided solely for their disclosure prior to the filing date of the present application. Nothing herein is to be construed as an admission that the inventor is not entitled to antedate such disclosure by virtue of prior invention.

Signature Conserved Sequences in Microorganisms

In recent years, microbial geneticists have sequenced a number of important human pathogens, and this information is readily available in the public domain. To date, about thirty different pathogens have been fully sequenced, and scientists are in the process of sequencing many additional microorganisms.

By analyzing this genetic information, the inventor has determined that there are important sets of protein/DNA sequences that are highly conserved among different pathogens. The genes in question code for proteins involved in essential cellular processes, such as for example, chromosome partition, cell division, genes associated with pathogenicity, cell wall proteins, and other functions easily identifiable by those skilled in the art. One conserved sequence, for example, is the FtsK/SpoIIIE gene, which codes for a product that has proved to be essential for bacterial chromosome partition.

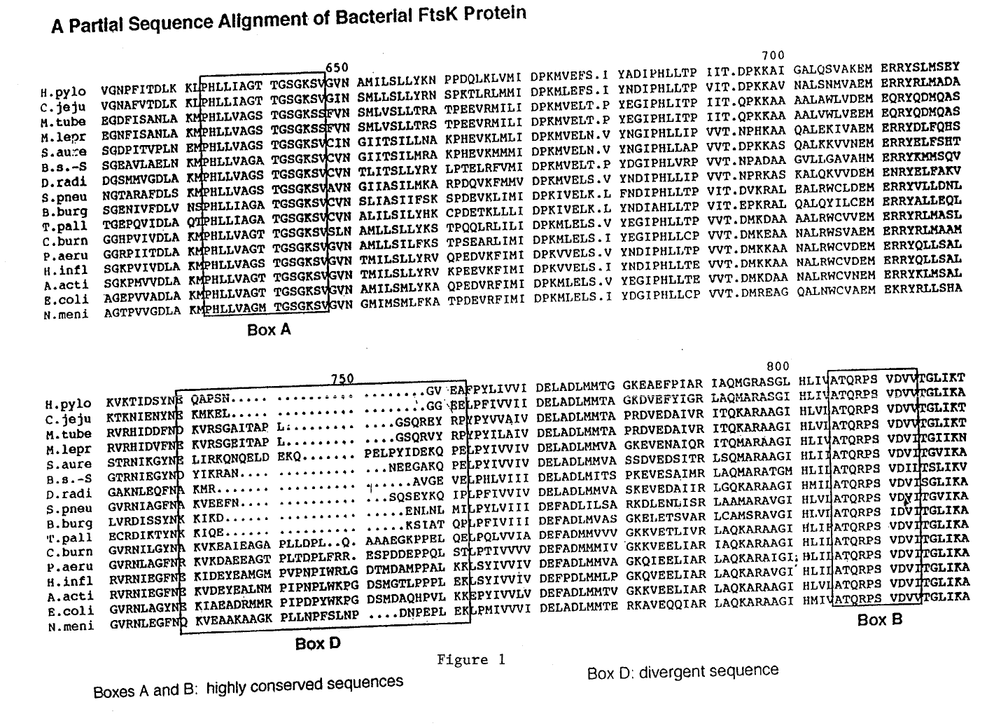

FIG. 1 shows a partial sequence alignment of FtsK proteins from various bacteria. Pair-wise comparison shows that these bacteria have about 50-70% sequence homology. Moreover, further analysis reveals that these coding sequences are conserved only in eubacteria; they are absent in archaebacteria and eukaryote genomes, reflecting the fact that chromosome partition/segregation in archaebacteria and eukaryotic organisms is mediated through different mechanisms. Thus, the FtsK coding sequence would be useful as a signature probe for bacterial pathogens. Other coding sequences (such as FtsZ, FtsQ, topoisomerases, tRNA synthetases, etc.) as well as several conserved non-coding sequences (such as, for example rDNA) can also be used as signature probes since degenerate PCR primers can be designed to amplify these sequences. The foregoing conserved sequences are provided by way of example only, other conserved sequence can be readily identified and are applicable to the methodology and compositions described herein, as discussed below.

The rationale for choosing highly conserved coding sequence to design the PCR primers is to simplify, for example, the diagnosis procedure in a clinical setting, where reliability and reproducibility are major concerns. For a given infectious disease and a particular patient, symptoms are often caused by one, out of many possible, etiologic agents. The challenge is to design a single PCR reaction that can reliably amplify a nucleic acid (i.e., DNA or RNA) sample from anyone of these possible pathogens for further analysis, such as, for example, by reverse dot blot hybridization. Selecting PCR primers for highly conserved coding sequences make this possible, although a mixture of degenerate primers may be used in place of a single primer as the number of pathogens to be surveyed increases.

PCR amplification with degenerate primers is widely used in academia to clone conserved genes from a new organism based on a known protein sequence (Rose et al, 1998). The term “degenerate primer” used herein means, for example, introducing mixed nucleotides at one or more positions into the primer to account for possible coding sequence variations as a result of the degeneracy of the genetic code. For pathogen identification, the coding sequences chosen to be analyzed are typically known or have been determined first. Consequently, a much better design of the degenerate primer pair is to use an equimolar mixture (or, the two degenerate primers of the pair in a defined ratio) of the actual coding sequences from the pathogen(s) to be surveyed that correspond to the same conserved peptide sequence.

One advantage of this system is a significant reduction in primer degeneracy, compared to the design of introducing mixed nucleotides at multiple positions and thus, less complication for the PCR reaction. The latter design is viable when the number of pathogens to be covered by the assay is low. Another advantage of this system is to enable one to normalize the rates of individual PCRs in the course of a multiplex reaction.

Identification of Signature Sequences of Microorganisms

There are several ways to identify the presence of a particular polynucleotide sequence in an organism of interest. For example, nucleic acid sequence-specific hybridization pioneered by Southern (Southern, 1975) allows highly specific detection of a particular polynucleotide sequence in an extracted DNA sample. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. An example of progressively higher stringency conditions is as follows: 0.2×SSC/0.1% SDS at about room temperature (low stringency conditions); 0.2×SSC and 0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically by one skilled in the art.

In addition, amplification of polynucleotide sequence by, for example, the polymerase chain reaction technique (PCR) developed by Mullis et al. (Saiki et al., 1986) can serve the same purpose. By properly choosing the primers, one can obtain amplified product of an expected size after a certain plurality of PCR cycles if the target sequence is present in the extracted sample containing nucleic acids or genetic material. This method offers great sensitivity, since a 30-cycle reaction can, in principle, generate an amplification on the order of 10⁹. In practice, the validity of the PCR product needs to be confirmed through other analyses, such as RFLP or Southern blot.

In one aspect of the invention, the PCR reaction amplifies the target sequence from a clinical sample, although the primer hybridization and the subsequent amplification provide specificity to some extent (i.e., only amplifying genetic material from the pathogens). The identification of the pathogens with high specificity derives from sequence specific hybridization, by choosing hybridization probes from the sequences flanked by the PCR primers. Each probe has an exact match in a particular pathogen. Due to codon bias, the nucleotide sequence corresponding to a conserved protein sequence varies among pathogens. This fact allows one skilled in the art to easily design probes that are sufficiently different from each other in such a way that only one probe hybridizes, under stringent conditions, to the PCR product amplified from a particular pathogen. Recent advances in microarray technology make hybridization to multiple probes a relatively easier task.

In another aspect of the invention, the hybridization probe(s) is spotted in discrete areas on a biochip, to streamline the hybridization process. This approach is very useful in a clinical setting if the biochip has a built-in sensor array, with each probe corresponding to a sensor. The sensor array will record and store the hybridization signals, which can be retrieved later, or in real-time, with other conventional devices, such as a desktop computer.

Non-natural analogs of nucleic acids may also be used as the probes. One example is peptide nucleic acid (“PNA”; Nielsen et al, 1991). PNAs are nucleic acid analogs with an achiral polyamide backbone consisting of N-(2-aminoethyl)glycine units replacing the phosphodiester linkages. The purine or pyrimidine bases are linked to each unit via a methylene carbonyl linker. PNAs are resistant to enzymatic degradation and hybridize to complementary nucleic acid sequences with higher affinity than analogous DNA oligomers. The hybridization follows Watson-Crick base-pairing rules (Soomets et al, 1999). Within the framework of the present invention, PNA probes can be used in place of the DNA probes described above. In fact, PNAs have been exploited as an alternative for making biochips in an array format (Weiler et al, 1997). In light of this example, other possible nucleic acid analogs may also be used as probes so long as they hybridize to the target nucleic acids in a sequence specific manner.

Amplification by PCR

As used herein, the term “amplifying” refers to increasing the number of copies of a specific polynucleotide. For example, polymerase chain reaction (PCR) is a method for amplifying a polynucleotide sequence using a polymerase and two oligonucleotide primers, one complementary to one of two polynucleotide strands at one end of the sequence to be amplified and the other complementary to the other of two polynucleotide strands at the other end. Because the newly synthesized DNA strands can subsequently serve as additional templates for the same primer sequences, successive rounds of primer annealing, strand elongation, and dissociation produce rapid and highly specific amplification of the desired sequence. PCR also can be used to detect the existence of the defined sequence in a DNA sample.

In general, the primers used for PCR amplification according to the method of the invention embrace oligonucleotides of sufficient length and appropriate sequence that provides initiation of polymerization of a significant number of nucleic acid molecules containing the target nucleic acid under the conditions of stringency for the reaction utilizing the primers. In this manner, it is possible to selectively amplify polynucleotides for further analysis. Specifically, the term “primer” as used herein refers to a sequence comprising two or more deoxyribonucleotides or ribonucleotides, preferably at least eight, which sequence is capable of initiating synthesis of a primer extension product that is capable of hybridizing to a target nucleic acid strand in order to initiate polymerase activity. The oligonucleotide primer typically contains 15-22 or more nucleotides, although it may contain fewer nucleotides so long as the primer is of sufficient specificity to allow essentially only the amplification of the desired target nucleotide sequences (e.g., the primer is substantially complementary).

Experimental conditions conducive to synthesis include the presence of nucleoside triphosphates and an agent for polymerization, such as DNA polymerase, and a suitable temperature and pH. The DNA polymerase is preferably a thermostable DNA polymerase, such as Taq polymerase, TthI polymerase, VENT polymerase or Pfu polymerase. The primer is preferably single stranded for maximum efficiency in amplification, but may be double stranded. If double stranded, the primer is first treated to separate the strands before being used to prepare extension products. Preferably, the primer is an oligodeoxyribonucleotide. The primer must be sufficiently long to prime the synthesis of extension products in the presence of the inducing agent for polymerization. The exact length of primer will depend on many factors, including temperature, buffer, and nucleotide compound.

Primers used according to the method of the invention are designed to be “substantially” complementary to each strand of a target nucleotide sequence to be amplified. Substantially complementary means that the primers must be sufficiently complementary to hybridize with their respective strands under conditions that allow the agent for polymerization to function. In other words, the primers should have sufficient complementarity with the flanking sequences to hybridize therewith and permit amplification of the nucleotide sequence. Typically, the 3′ terminus of the primer that is extended has perfectly base paired complementarity with the complementary flanking strand.

Oligonucleotide primers used according to the invention are employed in any amplification process that produces increased quantities of target nucleic acid. Typically, one primer is complementary to the negative (−) strand of the nucleotide sequence and the other is complementary to the positive (+) strand. Annealing the primers to denatured nucleic acid followed by extension with an enzyme, such as the large fragment of DNA Polymerase I (Klenow) or Taq DNA polymerase and nucleotides or ligases, results in newly synthesized + and − strands containing the target nucleic acid. Because these newly synthesized nucleic acids are also templates, repeated cycles of denaturing, primer annealing, and extension results in exponential production of the region (i.e., the target mutant nucleotide sequence) defined by the primer. The product of the amplification reaction is a discrete nucleic acid duplex with termini corresponding to the ends of the specific primers employed. The terms “forward” and “reverse” primers are interchangeable and used to define any one of a pair of related primers useful for the amplification of a target segment between the two primers. Those of skill in the art will know of other amplification methodologies that can also be utilized to increase the copy number of target nucleic acid.

Accordingly, as part of the invention, primers are designed that correspond to highly conserved regions of the genome of a family or a genus of organisms. In one embodiment, these primers are selected from regions of the genome that code for conserved proteins. These primers can be degenerate depending upon the sequence homology of the target polynucleotide to be amplified. Typically the primers flank a region of a gene (i.e., the target polynucleotide sequence) that is not highly conserved across species. Thus, during amplification of a sample containing genetic material, target polynucleotides will be amplified, for example, by PCR and the resulting PCR product then further analyzed as described more fully below.

“Genetic material” is a material containing any nucleic acid (DNA or RNA) sequence or sequences either purified or in a native state such as a fragment of a chromosome or a whole chromosome, either naturally occurring or synthetically or partially synthetically prepared nucleic acid sequences, nucleic acid sequences which constitute a gene or genes and gene chimeras, e.g., created by ligation of different nucleic acid sequences.

“DNA sequence” is a sequence of a linear or circular DNA molecule comprised of any combination of the four DNA monomers, i.e., nucleotides of adenine, guanine, cytosine and thymine, which codes for genetic information, such as a code for an amino acid, a promoter, a control or a gene product. A specific DNA sequence is one that has a known specific function, e.g., codes for a particular polypeptide, a particular genetic trait, or affects the expression of a particular phenotype. “Gene” is the smallest, independently functional unit of genetic material that codes for a protein product or controls or affects transcription and comprises at least one DNA sequence. A “coding sequence” is a polynucleotide sequence that is transcribed and/or translated into a polypeptide.

Specific Hybridization to Microarrays

In general, Southern techniques and PCR can be used to identify particular genomic sequences. In addition, recent developments in DNA chip (i.e., biochip) technology provide a third alternative. In essence, the DNA chip is a streamlined version of dot-blot analysis, a variation of Southern's method. Through miniaturization, a large number of probe sequences are deposited onto the surface of a solid support. The identity of the target sequence is defined by its specific hybridization to a probe or probes on the chip. The main advantage of this method is that it can survey a large number of probes with relative ease.

Accordingly, in one embodiment, oligonucleotides probes are immobilized to a solid support at defined locations (i.e., known positions). This immobilized array is sometimes referred to as a “biochip.” The solid support can be, for example, a nylon (polyamide) membrane, glass slide, silicon chip, polymer, plastic, ceramics, metal, optical fiber or other material. The solid support can also be coated (e.g., with gold or silver) to facilitate attachment of the oligonucleotides to the surface of the solid support. Any of a variety of methods known in the art may be used to immobilize oligonucleotides to a solid support. A commonly used method consists of the non-covalent coating of the solid support with avidin or streptavidin and the immobilization of biotinylated oligonucleotide probes. The oligonucleotides can also be attached directly to the solid supports by epoxide/amine coupling chemistry. See Eggers et al. Advances in DNA Sequencing Technology, SPIE conference proceedings (1993). By oligonucleotide probes is meant nucleic acid sequences complementary to a species-specific target sequence.

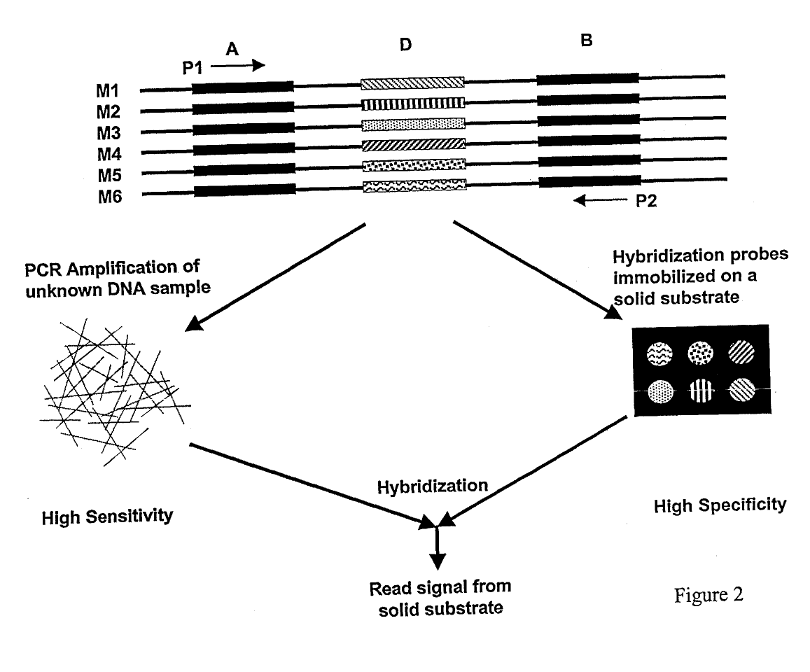

As schematically illustrated in FIG. 2, the PCR products are detected and distinguished by use of “biochips.” The chips are designed to contain probes exhibiting complementarity to a particular reference sequence from an organism of interest (e.g., viral, prokaryotic, eukaryotic). Typically, the probes present on the chip are sequences flanked by the degenerate PCR primers P1 and P2. The chips are used to read a target sequence comprising either the reference sequence itself or variants of that sequence representing the various species specific amplification products or target sequences. The sequence selected as a reference sequence can be from anywhere in the target organism with the proviso that they are flanked by the degenerate PCR primers P1 and P2 to the sequences A and B of the particular species or organism as shown in FIG. 2. A reference (e.g, probe) sequence is usually about 5, 10, 20, 50, 100, 5000, 1000, 5,000 or 10,000 bases in length, and typically about 20-2000 bases in length. The reference sequence can contain the entire region coding for the target sequence of interest or a fragment thereof. Various densities of the reference sequence may be present on the chip such as, for example, about 2 to more than 10,000 probe sequences/cm² or more (e.g., 100,000 probe sequence/cm²) typically about 10 to less than 1,000 probe sequences/cm².

Although the array of probes is usually laid down in rows and columns, such a physical arrangement of probes on the chip is not essential. Provided that the spatial location of each probe in an array is known, the data from the probes can be collected and processed to yield the sequence of a target irrespective of the physical arrangement of the probes on a chip. In processing the data, the hybridization signals from the respective probes can be reasserted into any conceptual array desired for subsequent data reduction whatever the physical arrangement of probes on the chip.

The length of probe can be important in distinguishing between a perfectly matched probe and probes showing a single-base mismatch with the target sequence. The discrimination is usually greater for short probes. Shorter probes are usually also less susceptible to formation of secondary structures. However, the absolute amount of target sequence bound, and hence the signal, is greater for larger probes. The probe length representing the optimum compromise between these competing considerations may vary depending on inter alia the GC content of a particular region of the target DNA sequence. In some regions of the target, short probes (e.g., 11 mers) may provide information that is inaccessible from longer probes (e.g., 19 mers) and vice versa. Maximum sequence information can be read by including several groups of different sized probes on the chip as noted above. However, for many regions of the target sequence, such a strategy provides redundant information in that the same sequence is read multiple times from the different groups of probes. Equivalent information can be obtained from a single group of different sized probes in which the sizes are selected to optimize readable sequence at particular regions of the target sequence. The strategy of customizing probe length within a single group of probe sets minimizes the total number of probes required to read a particular target sequence. This leaves ample capacity for the chip to include probes to other reference sequences (e.g., sequences of another conserved genomic region) as discussed herein.

Some chips may contain additional probes or groups of probes designed to be complementary to a second reference or target sequence. Although adding an additional set of probes for the same group of pathogens may seem redundant, it will help to ensure the reliability of whole process, in case the first set of probes fail to yield hybridization signals. Moreover, the second reference or target sequence can be a control sequence to determine accuracy of the amplification reaction or a control sequence to measure or quantitate the amount of target sequence in a sample. The process and principal of analysis for this secondary sequence is the same as that for the initial or target sequence.

The total number of probes on the chips depends on a number of factors, including the number of potential organisms to be identified, the length of the reference sequence and the options selected with respect to inclusion of multiple probe lengths and secondary groups of probes to provide confirmation of the assay.

The target polynucleotide or target genetic material, whose sequence or identity is to be determined, is usually isolated, in the case of therapeutic diagnostics, from a clinical fluid (e.g., urine, blood, plasma, sputum, cerebrospinal fluid, tracheal aspirate or pleural fluid) or tissue sample in the form of RNA or DNA. The RNA can be reverse transcribed to DNA, and the cDNA product then amplified by techniques known to those of skill in the art. Accordingly, in one embodiment target polynucleotides are prepared by PCR amplification in the presence of labeled nucleoside triphosphates. The resulting PCR products are hybridized under appropriate conditions to a probe sequence on a biochip and the unhybridized material washed away with buffer. The chip is subsequently scanned by autoradiography or in real time to determine the presence of hybridized product at particular locations on the biochip. A hybridized product is indicative of the presence of a microorganism corresponding to the probe sequence located on the biochip. When the target strand is prepared in single-stranded form as in preparation of target RNA, the sense of the strand should of course be complementary to that of the probes on the chip. This is achieved by appropriate selection of primers.

Diagnostic Applications

Bacterial sepsis and related septic shock are frequently lethal conditions caused by infections which can result from certain types of surgery, abdominal trauma and immune suppression related to cancer, transplantation therapy or other disease states. It is estimated that over 700,000 patients become susceptible to septic shock-causing bacterial infections each year in the United States alone. Of these, 160,000 actually develop septic shock, resulting in 50,000 deaths annually.

Gram-negative bacterial infections comprise the most serious infectious disease problem seen in modem hospitals. Two decades ago, most sepsis contracted in hospitals was attributable to more acute gram positive bacterial pathogens such as Staphylococcus and Streptococcus. By contrast, the recent incidence of infection due to gram-negative bacteria, such as Escherichia coli and Pseudomonas aeruginosa, has increased.

Gram-negative bacteria now account for some 200,000 cases of hospital-acquired infections yearly in the United States, with an overall mortality rate in the range of 20% to 60%. The majority of these hospital-acquired infections are due to such gram-negative bacilli as E. coli (most common pathogen isolated from patients with gram negative sepsis), followed in frequency by Klebsiella pneumoniae and P. aeruginosa.

Gram-negative sepsis is a disease syndrome resulting from the systemic invasion of gram negative rods and subsequent endotoxemia. The severity of the disease ranges from a transient, self-limiting episode of bacteremia to a fulminant, life threatening illness often complicated by organ failure and shock. The disease is often the result of invasion from a localized infection site, or may result from trauma, wounds, ulcerations or gastrointestinal obstructions. The symptoms of gram-negative sepsis include fever, chills, pulmonary failure and septic shock (severe hypotension).

Gram-negative infections are particularly common among patients receiving anticancer chemotherapy and immunosuppressive treatment. Infections in such immuno-compromised hosts characteristically exhibit resistance to many antibiotics, or develop resistance over the long course of the infection, making conventional treatment difficult. The ever-increasing use of cytotoxic and immunosuppressive therapy and the natural selection for drug resistant bacteria by the extensive use of antibiotics have contributed to gram-negative bacteria evolving into pathogens of major clinical significance.

The Gram-negative bacteria are a diverse group of organisms and include Spirochetes such as Treponema and Borrelia, Gram-negative bacilli including the Pseudomonadaceae, Legionellaceae, Enterobacteriaceae, Vibrionaceae, Pasteurellaceae, Gram-negative cocci such as Neisseriaceae, anaerobic Bacteroides, and other Gram-negative bacteria including Rickettsia, Chlamydia, and Mycoplasma.

Gram-negative bacilli (rods) are important in clinical medicine. They include (1) the Enterobacteriaceae, a family that comprises many important pathogenic genera, (2) Vibrio, Campylobacter and Helicobacter genera, (3) opportunistic organisms (e.g., Pseudomonas, Flavobacterium, and others) and (4) Haemophilus and Bordetella genera. The Gram-negative bacilli are the principal organisms found in infections of the abdominal viscera, peritoneum, and urinary tract, as well secondary invaders of the respiratory tracts, burned or traumatized skin, and sites of decreased host resistance. Currently, they are the most frequent cause of life threatening bacteremia. Examples of pathogenic Gram-negative bacilli are E. coli (diarrhea, urinary tract infection, meningitis in the newborn), Shigella species (dysentery), Salmonella typhi (typhoid fever), Salmonella typhimurium (gastroenteritis), Yersinia enterocolitica (enterocolitis), Yersinia pestis (black plague), Vibrio cholerae (cholera), Campylobacter jejuni (enterocolitis), Helicobacter jejuni (gastritis, peptic ulcer), Pseudomonas aeruginosa (opportunistic infections including burns, urinary tract, respiratory tract, wound infections, and primary infections of the skin, eye and ear), Haemophilus influenzae (meningitis in children, epiglottitis, otitis media, sinusitis, and bronchitis), and Bordetella pertussis (whooping cough). Vibrio is a genus of motile, Gram-negative rod shaped bacteria (family Vibrionaceae). Vibrio cholerae causes cholera in humans; other species of Vibrio cause animal diseases. E. coli colonize the intestines of humans and warm blooded animals, where they are part of the commensal flora, but there are types of E. coli that cause human and animal intestinal diseases. They include the enteroaggregative E. coli (EaggEC), enterohaemorrhagic E. coli (EHEC), enteroinvasive E. coli (EIEC), enteropathogenic E. coli (EPEC) and enterotoxigenic E. coli (ETEC). Uropathogenic E. coli (UPEC) cause urinary tract infections. There is also neonatal meningitis E. coli (NMEC). Apart from causing similar infections in animals as some of the human ones, there are specific animal diseases including: calf septicaemia, bovine mastitis, porcine oedema disease, and air sac disease in poultry.

The pathogenic bacteria in the Gram-negative aerobic cocci group include Neisseria, Moraxella (Branhamella), and the Acinetobacter. The genus Neisseria includes two important human pathogens, Neisseria gonorrheae (urethritis, cervicitis, salpingitis, proctitis, pharyngitis, conjunctivitis, pharyngitis, pelvic inflammatory disease, arthritis, disseminated disease) and Neisseria meningitides (meningitis, septicemia, pneumonia, arthritis, urethritis). Other Gram-negative aerobic cocci that were previously considered harmless include Moraxella (Branhamella) catarrhalis (bronchitis and bronchopneumonia in patients with chronic pulmonary disease, sinusitis, otitis media) has recently been shown to be an common cause of human infections.

The Neisseria species include N. cinerea, N. gonorrhoeae, N. gonorrhoeae subspecies kochii, N. lactamica, N. meningitidis, N. polysaccharea, N. mucosa, N. sicca, N. subflava, the asaccharolytic species N. flavescens, N. caviae, N. cuniculi and N. ovis. The strains of Moraxella (Branhamella) catarrhalis are also considered by some taxonomists to be Neisseria. Other related species include Kingella, Eikenella, Simonsiella, Alysiella, CDC group EF-4, and CDC group M-5. Veillonella are Gram-negative cocci that are the anaerobic counterpart of Neisseria. These non-motile diplococci are part of the normal flora of the mouth.

Specific E. coli phenotypes have been associated with intestinal diseases, notably diarrhoea, and extraintestinal conditions including urinary tract infections and meningitis in the newborn. Like many pathogens, E. coli strains produce adhesins structures that mediate attachment to eukaryotic cells and which can be distinguished by their specificity for receptors on the target cell. Adhesins can represent the filamentous, hair-like structures known as fimbriae or pili, or they may be nonfilamentous components of the cell surface. Common F1A (type 1) fimbrial adhesins recognize the sugar a-mannose in glycoproteins, whereas mannose-resistant (MR) adhesins bind to eukaryotic receptors other than mannose. A wide range of filamentous adhesins are produced by different E. coli strains with specificities for various receptors on human and animal tissues. Pathogenic strains may contain sets of genes encoding one or more types of fimbriae, sometimes in combination with nonfimbrial adhesins.

Besides testing pathogens in a clinic sample, this invention can also be used to test food-borne bacteria, such as E. coli and Salmonella etc. Such safety measures will reduce the actual number of infections caused by food-borne pathogens.

Selection of Probes

For detection of clinical pathogens, combining PCR and Southern blot (e.g., a dot blot version or “biochip” technology) provides both sensitivity and specificity (or accuracy), both of which are essential for clinical testing. Currently, 16S rDNA and 23S rDNA have been used as the target sequence for PCR amplification (these sequences encode ribosomal RNA rather than protein, and they are highly conserved at the nucleotide level). One can easily design a set of primers that would work on genomic DNA from many different microbial pathogens. However, the subsequent Southern blot analysis would be less informative due to cross-species hybridization. For a clinical test, the ideal genomic regions are highly conserved coding sequences (for designing the PCR primers) flanking a less conserved coding sequence (for designing the hybridization probe). In principle, conserved non-coding regions, such as 16S rDNA, can also be used for this kind of analysis, except that greater efforts are required to eliminate possible artifacts.

The following advantages of using conserved protein coding sequences for diagnostic assay in a microarray format are significant in the selection of signature probes for a microorganism. Firstly, use of conserved protein coding sequences results in a different type of diagnostic test than comparable ribosomal DNA based approach. While there are many different protein families with varying degrees of amino acid sequence conservation, it is conceivable that in some cases one would use highly conserved protein coding sequence for diagnostic purpose, while in other cases, it would be preferable to use less conserved protein coding sequences. For example, among the 12 recognized serogroups of Neisseria meningitis, a less conserved protein coding locus would be preferred than a highly conserved protein coding sequence in order to ensure sufficient sequence differences to allow intra species distinction. On the other hand, the rDNA loci (and the corresponding intergenic region) appears to be too highly conserved in sequence to be useful for DNA-chip based diagnosis in this case.

Another important criteria for a good diagnostic assay is its accuracy. The built-in redundancy generated by using two or more independent loci for identification enables one to achieve better accuracy. The present invention allows the selection of multiple target sequences, from hundreds of conserved protein coding sequences in a microorganism, to be used in a single diagnostic test.

Conserved coding sequences are selected such that they are highly conserved at both ends of an operationally defined gene fragment and more divergent in the intervening coding sequence. For example, a preliminary analysis of FtsZ gene suggests that it has a high degree of conservation throughout. Type I and Type II topoisomerases are also examples of highly conserved genes in prokaryotes and eukaryotes. For a given organism, these functions are often encoded by multiple genes that share sequence similarity. Whereas these properties make them less preferred for application in the present invention, segments of these genes may still be suitable for the purposes of this application.

Whereas the invention discloses several unique probe sequences, an oligonucleotide comprising any 5 uninterrupted nucleotides in a disclosed probe sequence is suitable for the application of this invention. The term “probe” as used herein is thus intended to encompass any 5 uninterrupted nucleotides of a specific claimed or disclosed probe sequence.

In another embodiment of the present invention, an “universal primer” is used to amplify the target sequence, followed by sequencing of the amplified target. Comparison of this sequence with known sequence data enables the identification of the microorganism. In fact, the bacterial rDNA locus has been utilized in this fashion (e.g., in ribotyping). A variation of this scheme is to determine the sequence of the amplified sequence by on-chip hybridization to a high-density oligonucleotide microarray (as described in U.S. Pat. Nos. 5,202,231 and 5,002,867, incorporated herein by reference).

Effect of Primer Concentration

The present invention encompasses creation of an “universal primer” by mixing together related primers. It differs from conventional multiplex PCR primers in that all the primer pairs amplify the same genetic locus, albeit from different organisms. It also differs the conventional degenerate PCR primers which incorporate mixed base(s) at certain position(s) on the primer during its chemical synthesis. The advantage of mixing a number of primers of specific sequences over a single degenerate primer is two fold. One is to significantly reduce degeneracy of the primer. The other is to allow normalization of the individual reaction rates by adjusting the corresponding primer concentrations. The point is illustrated in the following example. Sequences of primers for the RecA gene of 11 different microorganisms and a degenerate consensus sequences are shown in the following table:

TABLE 1

Aligned sequences of RecA primers and a
consensus sequence.

| | |
|---|---|
| Ecoli | GGAATCTTCCGGTAAAACCAC |
| Bfrag | GGAATCATCCGGTAAAACGAC |
| Efaec | TGAGAGTTCAGGTAAAACAAC |
| ChlyP | CTGAATCCTCAGGGAAAACGAC |
| Spneu | AGAGTCATCTGGTAAGACAAC |
| Saure | TGAAAGTTCTGGTAAGACAAC |
| Mpneu | GAGTCCTCGGGTAAAACCAC |
| Hinfl | CTGAATCATCGGGTAAAACAAC |
| Lpneu | AGTCCTCGGGTAAAACCAC |
| PseuA | AATCCTCGGGCAAGACCAC |
| Kpneu | GAATCCTCCGGTAAAACCAC |
| | A GAGC A C G C |
| Consensus | 5′-CTGAATCATCGGGTAAAACAAC-3′ |
| | G T C G G |
| | T |

The degeneracy of the primer designed according to conventional method is 3×2×2×2×3×4×3×2×3=5184. Of these more than 5000 sequences, only 11 match the intended target sequences. The other sequences may or may not contribute to the PCR. The net result is a decrease in the concentration of the specific primer(s) in the reaction and an increase in the probability for the occurrence of non-specific priming events. If the pathogens to be covered by the PCR include 40 or so bacteria, the degeneracy is too high to be practical or useful. On the other hand, the primer degeneracy based on the “universal primer” according to the present invention is only 11. In order to identify 40 different bacteria from a single PCR reaction a degeneracy of 40 is more reasonable.

As shown in the above example, the RecA primers have different lengths, varying at the 5′-end. This normalizes the melting temperature (Tm) of the primers, such that each corresponding PCR is performed at the same annealing temperature. In a preferred Tm normalization method, the 3′-end of a group of primers is determined and the 5′-end is extended according to the sequence until the primer reaches the desired Tm. Since proper annealing at the 3′ end of the primer is essential for the PCR, a preferred mode of the invention has four out of five bases matched at the 3′ end of the primers. This ensures that the primers are more compatible with each other for substitutions during priming, in spite of mismatch(es) on the 5′ ends. The requirement that the 3′ end of the primer starts at position corresponding to two highly conserved amino acids in the coding sequence can be easily determined.

Another aspect of the present invention ensures that each individual PCR proceeds at similar or comparable rate, to avoid possible “drop-outs.” In a multiplex PCR of two separate reactions, if one proceeds faster than the other, the likely result of a standard 35-cycle reaction would be the disappearance of the weaker reaction product (i.e. “drop-out”) from the final product. Such false-negative results are undesirable for a diagnostic test. The present invention allows further normalization of the reaction rate of each individual PCR by adjusting the concentration of the corresponding primer pair in the primer mixture. Since all primers are related, especially at the 3′-end, by design, a primer running low at the later cycles can be compensated by the others, achieving the effect of a single pair of “universal primers”.

### EXAMPLES

Without further elaboration, it is believed that one skilled in the art can, using the preceding description, utilize the present invention to its fullest extent. The following examples are illustrative only, and not limiting of the remainder of the disclosure in any way whatsoever.

### 1. Effect of Primer Concentration

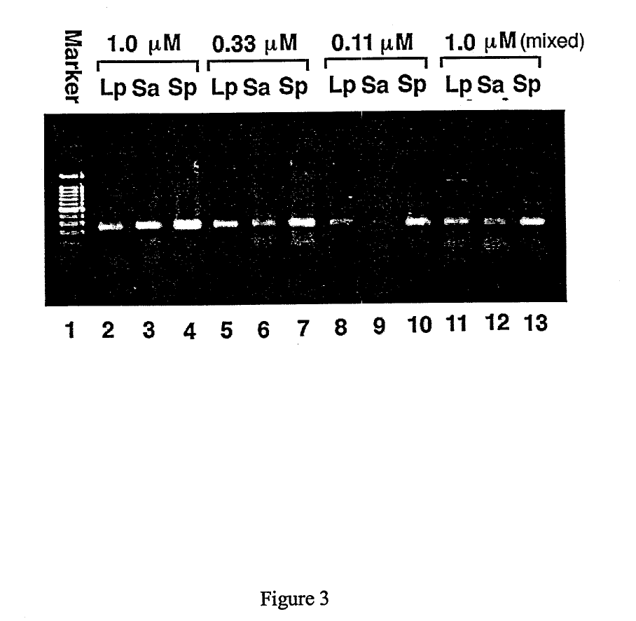

While there are multiple factors that affect the rate of a PCR reaction, the present invention exploits the effect of primer concentration. FIG. 3 shows the effect of primer concentration on amplification by individual set of PCR primers and mixed PCR primers for a RecA gene fragment. Standard 50 μl PCR reactions were carried out at various primer concentration, using genomic DNA from Legionella pneumonia (Lp), Staphylococcus aureus (Sa), and Streptococcus pneumonia (Sp) as the template. After a 27 cycle reaction, 10 μl aliquots were taken from each tube, resolved on a 2% agarose gel and then stained with ethidium bromide (EtBr). The DNA templates added to the PCR reaction were as follows: Legionella pneumonia, lane 2, 5, 8, and 11; Staphylococcus aureus, lane 3, 6, 9, and 12; Streptococcus pneumonia, lane 4,7, 10, and 13. Primer concentrations for the reactions were: lane 2-4, 1 μM of specific primer; lane 5-7, 0.33 μM specific primer; lane 8-10, 0.11 μM of specific primer; lane 11-13, 1.0 μM of mixed primers (an equimolar mixture of nine different pairs of primers, the effective concentration for a specific pair of primer being the same as in lanes 8-10). Lane 1 includes a 100 bp DNA size marker. The primers used were: Legionella pneumonia, SEQ 36 and 49; Staphylococcus aureus, SEQ 33 and 46; Streptococcus pneumonia, SEQ 32 and 45. Other primers that comprised the equimolar primer mixture were: SEQ ID NOS 35 and 48, 30 and 43, 34 and 47, 29 and 42, 31 and 44, 28 and 41.

For a PCR running with primers having similar Tm, at the same concentrations, the reaction rates are not the same, as shown in FIG. 3. However, for each PCR, the reaction rate can be controlled by adjusting the primer concentration as shown in FIG. 3. Although the primers have similar Tm, the reaction for S. aureus and L. pneumonia RecA are slower than that for S. pneumonia RecA. However, when an equimolar mixture of nine RecA primer pairs were used (a 9-fold dilution of each specific primer pair), some of the similar primers compensated for the decrease of specific primers (compare lanes 11 and 8; and lanes 12 and 9).

### 2. Comparison of PCR Rates Between Mixed Primers and Specific Primers

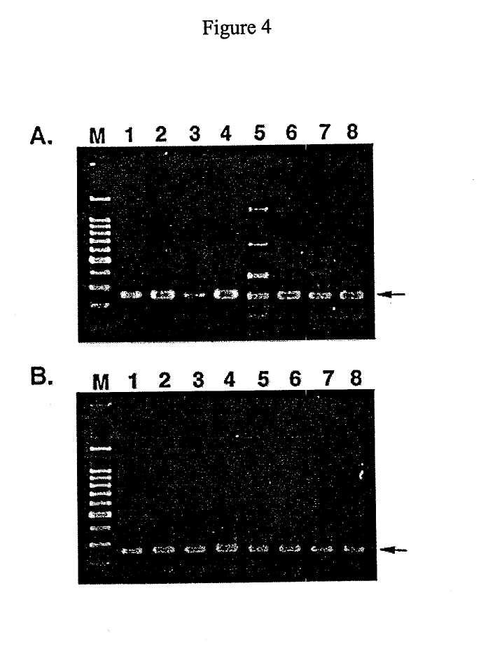

The reaction rates of a multiplex PCR can be normalized by mixing primer pairs at unequal molar ratios. FIG. 4A shows RecA PCR for eight different bacteria, using specific primers. It is evident that the reaction rates are different, even though the primers were normalized to a similar Tm (68 to 70° C.). When primers are mixed at the appropriate ratios (see Table 2), the reaction rates are normalized, as shown in FIG. 4B.

In the experiment the results of which are shown in FIG. 4, standard PCR were carried out using either specific primer pair (panel A) or mixed “universal primers”, under identical conditions. Following a 35-cycles reaction, the products were resolved on a 0.2% agarose gel. The templates used were 0.2 ng of genomic DNA from: lane 1, Enterococcus faecalis; lane 2, Bacteroides fragilis; lane 3, Staphylococcus aureus; lane 4, Haemophilus influenzae; lane 5, E. coli; lane 6, Legionella pneumophila; lane 7, Mycoplasma pneumoniae; lane 8, Streptococcus pneumoniae. Lane M is a 100 bp DNA size marker. The primers used for panel A were: lane 1, SEQ ID NOS 30 and 43; lane 2, SEQ ID NOS 29 and 42; lane 3, SEQ ID NOS 33 and 46; lane 4, SEQ ID NOS 35 and 48; lane 5, SEQ ID NOS 28 and 41; lane 6, SEQ ID NOS 36 and 49; lane 7, SEQ ID NOS 34 and 47; lane 8, SEQ ID NOS 32 and 45. The primers used for panel B were the same in all reactions, “universal primers” mixed according to the ratios shown in Table 2.

It is worth noting that the non-specific amplification products of E. coli PCR with a single primer pair (FIG. 4A, lane 5) were absent in a similar reaction with the mixed “universal primers” (FIG. 4B, lane 5). While this result is somewhat unexpected it is of practical utility. The results can be rationalized by considering that the E. coli primers may anneal to loci other than FtsY and generate the non-specific amplification products. However, when mixed “universal primers” were used, two factors contributed to the disappearance of the non-specific priming events. One, the E. coli primers were diluted significantly reducing their ability to anneal to other loci. The second is that other primers in the reaction, thought slightly different in their sequences, compensated for the decreased annealing of E. coli primers at FtsY locus, but did not anneal to other loci, due to sequence differences.

One method of achieving the proper primer mixing ratio is to titrate each specific primer pair, by dilution, in a linear reaction (e.g. 25 cycles PCR) and then, select the primer concentration for each specific primer pair that gives a comparable reaction rate to the others.

TABLE 2
Mixing ratio of primer pairs from eight different bacteria

| Organism | SEQ ID NO | Mixing ratio |
|---|---|---|
| S. pneu | 32, 45 | 1 |
| H. infl | 35, 48 | 0.5 |
| L. pneu | 36, 49 | 2 |
| B. frag | 29, 42 | 2 |
| M. pneu | 34, 47 | 4 |
| E. coli | 28, 41 | 8 |
| S. aure | 33, 46 | 16 |
| E. faec | 30, 43 | 16 |

### 3. Mutation Disrupting 3′ End Hair-Pin Formation in a Primer

The original primer pair designed for S aureus FtsY were 5′-TGTGAATGGTGtTGGTAAAACAAC-3′ (derived from wild type S. aureus FtsY gene sequence; SEQ ID NO 10 is a mutated version of this primer in which the “t” is changed to “A”) and 5′-TTTGTAAACGTCCAGCGGTATC-3′ (SEQ ID NO 23 is wild type sequence). When used in a standard PCR, these produced surprisingly low yields (data not shown).

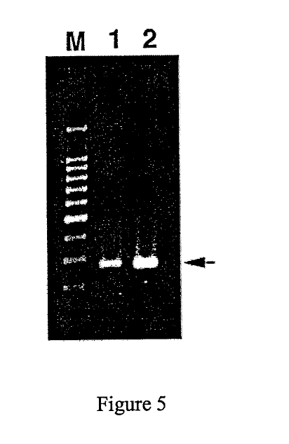

Sequence analysis suggests that the first primer can form a hair-pin structure (bases in bold-type), in which the four bases at the 3′ end fold back and form a 4 base-pair stem. An expected conclusion from this interpretation is that disruption of the hair-pin formation should increase the reaction rate. This is indeed the case, as shown in FIG. 5 (lane 3). When the mutated forward primer was used, where a internal T (designated by the lower case t) was changed to A, the PCR generated more products.

PCR experiments using a mutation that disrupts 3′-end hair-pin formation in a primer for S. aureus FtsY gene is shown in FIG. 5. Standard PCRs were carried out for S. aureus FtsY, using different primer pairs. The PCR products were resolved on a 2% agarose gel, stained with EtBr. The same backward primer (SEQ ID NO 23) was used for both reactions, but the forward primer was: lane 2, primer derived from wild type sequence; lane 3, mutated primer (i.e., SEQ ID NO 10).

There are at least two different interpretations on what may happen during PCR using the wild type forward primer. The first is that the hair-pin structure may self-prime at room temperature or the annealing temperature (i.e. 53° C.), extending the primer at the 3′ end. Although this product can anneal to the template at the original site, it cannot prime the intended PCR reaction due to lack of proper base-pairing at the 3′ end, thus becoming a competitive inhibitor. The other is that, during each PCR cycle, the hair-pin structure is disrupted at 92° C., yet a certain percentage refold at the annealing temperature, reducing the effective concentration of the forward primer. Both scenarios are consistent with the result shown in FIG. 5 that permanent disruption of hair-pin formation via mutations of the primer improves the PCR reaction.

In another aspect of the present invention, base modification of the primers to reduce secondary structures is performed. Because the general location of the primers on the target sequences is fixed for all the pathogens to be identified, such modification enables one to improve the weaker reactions without having to drastically change the primer sequences (e.g., generate primers from a different location on the target sequence).

### 4. Mycobacterial Identification

Genomic DNA samples from Mycobacterium tuberculosis and Mycobacterium leprae can be distinguished by nested PCR, followed by sequence specific hybridization. The same sets of primers can be used to amplify the FtsK gene fragment from either genomic DNA, because of the high degree of nucleotide sequence conservation at the chosen FtsK coding regions (a single nucleotide difference in some of the primers is indicated by a capital letter, below). The unknown DNA prepared from a clinic sample will be used as the template for the first PCR reaction, with the primer set of:

| | |
|---|---|
| 5′-aagtcCagcttcgtcaac-3′; and | (SEQ ID NO:1) |
| 5′-gccgtcGcccatgccgatca-3′. | (SEQ ID NO:2) |

After a standard 30-cycle reaction, an aliquot of the reaction product will be used as the template for the second PCR reaction with the primer set of: 5′-ccgcatCtgatcacgccgatcatc-3′ (SEQ ID NO:3) and 5′-acgtcGtccgacgggcgtag-3′ (SEQ ID NO:4) (both fall within the sequence amplified by SEQ ID NOS: 1 and 2, i.e., internal—3′ to SEQ ID NO:1 and 5′ to SEQ ID NO:2—set forth by the first set of primers, and one of the two will have a biotin label at the 5′ end). After another 30-cycle reaction, the PCR product will be used directly in a hybridization reaction, probing a Nylon membrane. The Nylon membrane is prepared in such a way that it has two discrete spots with different oligonucleotides attached to the membrane at the two spots, respectively. One oligonucleotide is 5′-atcgacgacttcaacgacaag-3′ (SEQ ID NO:5), derived from M. tuberculosis FtsK coding sequence (from Box D shown in FIG. 1). The other is from M. leprae FtsK, having the sequence of 5′-atcgacgTGttcaaCgagaag-3′ (SEQ ID NO:6). This sequence differs from the first oligonucleotide (i.e., SEQ ID NO:5) at three nucleotide positions (indicated in upper case). Under appropriate hybridization stringency, only one probe will hybridize to the PCR product, depending on the origin of the unknown DNA sample. The specific hybridization pattern can be revealed in a number of way, such as streptavidin conjugated alkaline phosphates, radionucleotide labeling followed by autoradiography or by chemiluminescence (Bronstein et al. 1990). Based on the hybridization result, one can determine the bacterial origin of the unknown DNA sample.

### 5. Bacterial Meningitis Identification

Meningitis can be viral or bacterial in origin, with the latter causing the more severe illness. Etiologic agents for bacterial meningitis are usually Neisseria meningitidis, Haemophilus influenzae, and Streptococcus pneumoniae. Currently, the identification of precisely which bacterium is the culprit requires lengthy laboratory tests. The present invention provides an alternative for rapid and accurate identification. Based on the FtsK coding sequence from these three bacteria, PCR primers and hybridization probes are designed as follows:

| | |
|---|---|
| N. menig | 5′-gcaccgcatttgttggttgccgg-3′ |
| H. influ | 5′-atgccacatttattggtagcagg-3′ |
| S. pneu | 5′-atgccccacyygctagttgcagg-3′ |

These oligonucleotide sequences are derived from the conserved coding region, Box A (FIG. 1). For doing actual PCR, an equal molar ratio mixture of these three oligonucleotides will be used, which is equivalent to a single primer with a three-fold degeneracy.

| | |
|---|---|
| N. menig | 5′-atgacatcgacactggggcgttg-3′ |
| H. influ | 5′-atcacatccacagaggggcgttg-3′ |
| S. pneu | 5′-atgacatcaacagatggacgctg-3′ |

These oligonucleotides are derived from the conserved Box B (FIG. 1). An equimolar mixture is used for the PCR reaction.

| | |
|---|---|
| N. menig | 5′-aaaatcgccgaagccgcagcaagg-3′ |
| H. influ | 5′-aaaattgatgaatacgaagcaatg-3′ |
| S. pneu | 5′-gaagagttcaattcccagtctgag-3′ |

These oligonucleotides are derived from the divergent coding region, Box D (FIG. 1). Each oligonucleotide is derivatized with Acrydite (Mosaic Technologies), which will allow them to be immobilized directly in a discrete spot on the surface of a glass slide (pretreated with acrylic silane).

The unknown DNA sample prepared from a clinical sample will be used as the template for the PCR reaction. A single PCR reaction will be performed using the degenerate Forward and Backward primer set (each primer is derivatized with a fluorescent dye during its chemical synthesis). After a standard PCR reaction, the products are hybridized with the probe panel in situ, followed by a brief wash. The hybridization pattern is then observed under a fluorescent microscope or a confocal microscope. The bacterial origin of the DNA sample is indicated by the probe that hybridized to the PCR product.

### 6. Unicellular Eukaryotes (Fungi) Identification

Identification of different eukaryotic cells can accomplished based on the same principle. Fungi are known etiologic agents that caused pneumonia, such as Aspergillus parasiticus and Candida albicans. Like other eukaryotes, these two different organisms share a number of highly conserved proteins that are involved in various essential cellular processes. For example, beta-tubulin protein is highly conserved both in sequence and function, i.e. mediating chromosome segregation during eukaryotic cell division. The human CDC2 protein is also highly conserved, with an essential function of regulating eukaryotic cell cycle progression. To illustrate the principle of this invention, a test based on beta-tubulin gene is described here, although other conserved protein coding sequences can also be used. The genomic sequences encoding beta-tubulin from Aspergillus parasiticus and Candida albicans are available from GenBank (Accession number L49386 and M19398 respectively). Different from the bacteria cases describe above, eukaryotic protein coding sequences are usually Interrupted by non-coding sequences, i.e., introns. For a given conserved gene, the number of introns as well as the location of the introns within the gene are not necessarily conserved. The beta-tubulin gene from C. albicans two introns, with the exon 3 encoding amino acids 17 to 449. That from A. parasiticus contains seven introns, with the exon 6 and exon 7 encoding amino acids 54 to 436. These two proteins share about 80% sequence identity or 90% sequence similarity. PCR primers are chosen from the conserved coding regions in the exon 3 for C. albicans, or from exons 6 and 7 for A. parasiticus. The nucleotide sequences are listed below.

| | |
|---|---|
| A. para | 5′-aagtatgtccctcgtgccgt-3′ |
| C. albi | 5′-aaatacgttcctcgtgccgt-3′ |

| | |
|---|---|
| A. para | 5′-ctccatctcgtccatacc-3′ |
| C. albi | 5′-ttccatttcatccatacc-3′ |

The DNA extracted from a clinic sample is used as the template for the first PCR reaction. An equal molar mixture of the forward primers as well as that of the backward primers are added to the standard amplification. After a 30-cycle reaction, an aliquot of the product is taken out and used as the template for the second round of amplification (i.e. nested PCR). The primers used for the second PCR reaction are flanked by the first pair of PCR primers, respectively. Hence, the second PCR reaction will further amplify the desired product, and offer an additional specificity check. Again, an equal molar mixture of the primers for these two organisms are used for the reaction to avoid possible bias for a particular pathogen. The actual sequences of the second pair of primers are listed below.

| | |
|---|---|
| A. para | 5′-ggtgccggtatgggtact-3′ |
| C. albi | 5′-ggttctggtatgggtact-3′ |

Second backward primers (each primer is biotinylated at the 5′ end):

| | |
|---|---|
| A. para | 5′-ggagtttccaataaaggt-3′ |
| C. albi | 5′-agagtttccaataaaagt-3′ |

After the second PCR reaction, the products are extracted once with phenol once, and hybridized to nylon membrane with a panel of immobilized probes. Two of the probes are from A. parasiticus and one from for C. albicans. All of them are flanked by the second set of PCR primers. Each probe is located within a restricted area of the membrane. The nucleotide sequences of the probes are:

| | |
|---|---|
| A. para-1 | |
| 5′-cgcaacatccagagcaagaaccagacc-3′ | |
| A. para-2 | |
| 5′-ttgtttgaaaactgacccttccatagc-3′ | (intron 6 probe) |
| C. albi | |
| 5′-cacaaaatccaaaccagaaactcatct-3′ | |

After hybridization and washing under stringent conditions, the hybridization pattern is display by alkaline phosphatase and chemiluminescent, followed by autoradiography. The specific hybridization to a particular probe indicates the genomic origin of PCR product, hence the identity of the pathogen in the clinic sample.

It should be emphasized that one of the hybridization probe (A. para-2) is chosen form Intron 6 sequence of A. parasiticus beta-tubulin gene. This intron (thus the hybridization probe sequence) is completely absent from PCR product amplified from for C. albicans genomic DNA. It may work better in terms of discriminating between the PCR products. This example illustrates that, when the present invention is applied to eukaryotic sample, the hybridization may be chosen from an intron sequence rather than a less conserved protein coding sequence.

### 7. Virus Identification

Virus is another major class of infectious agents. The present invention also provides a mean for the systematic detection of multiple pathogens from this class. Among viruses, certain proteins or functions are highly conserved. The replication of a viral genome is an essential step in the life cycle of the virus. It invariably requires the participation of at least a viral-encoded DNA or RNA polymerase. Within a subclass of viruses, these polymerase are usually conserved, due to evolutionary constrain on the replication function. For example, reverse transcriptase is highly conserved among retroviruses. In this embodiment, a method is described that detects and distinguishes a class of single stranded RNA viruses. In a clinic study (Ahn et al, 1999), it has been shown that viral etiologic agents for acute lower respiratory track infection in children include adenovirus (12.7% of the total viral isolates), influenza virus type A (21.1%), -type B (13.9%), parainfluenza virus type 1 (13.5% ), -type 2 (1.3%), -type 3 (16.0%) and respiratory syncytial virus (21.5%). Among the 237 patients studied, the overall viral isolation rate was 22.1%. Of these viruses, parainfluenza virus type 1 (PIV-1), -type 2 (PIV-2), -type 3 (PIV-3), and respiratory syncytial virus (RSV) belong to Paramyxoviridae family of enveloped negative-strand RNA viruses. Other members of this family also include Ebola virus, Newcastle disease virus, Sendai virus, Measles virus, and Hendra virus etc. Of the four viruses that cause respiratory-track infection, a single PCR reaction can be designed based on the conserved coding sequences within the RNA polymerase gene (L-protein), which will detect all four viruses. Because these are RNA viruses, a reverse transcriptase reaction will be needed to convert the interested genomic RNA into DNA for the PCR reaction. As an example, the coding sequences for amino acids 537 to 542 (IDKAIS) and amino acids 776 to 781 of RSV RNA polymerase (GenBank accession number U39662) are chosen as the PCR primers. The C-terminal primer (A.A. 775 to 781) will also be used as the primer for the reverse transcriptase reaction. Primers for the other viruses will be chosen from the corresponding coding sequences, based on protein sequences alignment. For PIV-3 (GenBank accession number U51116), the primers encode amino acids 497-502 and amino acids 763-768. For PIV-1 (GenBank accession number AF117818), the primers correspond to amino acids 472-477 and amino acids 738-743. For PIV-2 (GenBank accession number X57559), the primers correspond to amino acids 475-480 and 742-747. The actual sequences of these primers are listed below.

Forward primers (equal molar mixture):

| | |
|---|---|
| PIV-3 | 5′-atgaaagataaagcatta-3′ |
| PIV-1 | 5′-atgaaggataaggctcta-3′ |
| PIV-2 | 5′-atgaaagacaaggcaata-3′ |
| RSV | 5′-ataaatgataaggctata-3′ |

Backward primers: (equal molar mixture)

| | |
|---|---|
| PIV-3 | 5′-acaaaatccttctatacc-3′ |
| PIV-1 | 5′-gcaataaccttctattcc-3′ |
| PIV-2 | 5′-acataggccttcaatacc-3′ |
| RSV | 5′-acaccacccttcgatacc-3′ |

To perform a clinic test, nucleic acid sample extracted from nasopharyngeal aspirate of a patient will be used as the template. The backward primer pool (an equal molar mixture of the four primers, which is equivalent to a single primer with a four-fold degeneracy) is used to initiate the reverse transcriptase reaction first, according to standard reaction condition. Then, the forward primer pool (each of the primer has a biotin molecule derivatized at the 5′ end) is added to start the PCR reaction, following standard protocol. After a 30-cycle reaction, the PCR products are extracted once, and hybridized to a nylon membrane with a panel of immobilized hybridization probes. The exact sequences of the probes correspond to a stretch of non-conserved amino acid sequence of the RNA polymerase, flanked by the PCR primers. They are listed below.

Hybridization probes:

| | |
|---|---|
| PIV-3 | 5′-ttgtcttctaatcagaaatca-3′ |
| PIV-1 | 5′-aatgggtattgggatgaaaga-3′ |
| PIV-2 | 5′-aagactgattctaaaaataag-3′ |
| RSV | 5′-tacattagtaagtgctctatc-3′ |

Each probe is spotted onto the membrane in a separate discrete area, and cross-linked to the membrane by UV irradiation. These sequences are sufficiently different to allow the differentiation of specific hybridization to respective PCR products, under stringent conditions. After the hybridization and subsequent washes under stringent condition, the membrane is treated with streptavidin-alkaline phosphatase conjugate. The hybridization signal is reveal by adding chemiluminescent substrate followed by autoradiography. The specific hybridization of the PCR product to a particular viral probe indicates the presence of that virus in the clinic sample.

### 8. Pneumonia Pathogen Identification

The pathogens described in examples 2, 3, and 4 can all cause pneumonia. The fastest way, and the economic way, to identify the actual pathogen for a particular patient is to include all these pathogens in a single test, rather than separate tests. To achieve this objective, the hybridization probes described in above examples (including bacteria, viruses, and fungi) are spotted onto a single chip or a piece of nylon membrane, to obtain a disease-specific probe panel. For a particular clinic sample, the PCR reactions described above are carried out in parallel. The amplified products are pooled and hybridized to the disease-specific probe panel in a single step. The specific hybridization of the PCR products to the probe panel is indicated by fluorescence or chemiluminescence as described in previous examples. More pathogens can be included in this single test, based on the principles for the PCR primer and hybridization probe design disclosed herein. Furthermore, for other infectious diseases (such as STD), similar tests can be developed that include all the known etiologic agents in a single assay, based on the same principle.

### 9. Simultaneous Identification of Bacteria and Fungi

In one embodiment of the present invention, both prokaryotic and eukaryotic cells can be identified simultaneously in a single test, since many genes are highly conserved among them.

Candida albicans is a significant respiratory-track pathogen. A database search, using E. coli FtsY protein sequence as the query, easily identified its homologue in Candida albicans, and the yeast S. cerevisiae. These proteins share about 30% amino acid sequence identity and 50% DNA sequence identity with their prokaryotic counterpart over a stretch of 300 amino acids. The corresponding coding sequences were retrieved from GenBank and aligned with the prokaryotic FtsY coding sequences, using Clustal W program. PCR primers were designed based on the alignment, such that they were compatible with the bacterial FtsY primers. When standard PCR was performed with these new primer pairs individually, a single product of the expected size was amplified.

This result validate the underpinning principle that if PCR works well for a particular locus of one organism, it will also work on the same locus of a different organism using primers designed accordingly, so long as the locus is conserved. In the case of FtsY, the same primer design works for a number of bacteria, including mycoplasma (with a much smaller genome), and fungi (more complex genome).

Although the size of FtsY PCR products from fungi and bacteria are very similar, they can be easily distinguished by hybridization to specific probes for each organism, derived from the divergent regions flanked by the PCR primers. To validate this point, PCR was performed using a mixture of FtsY primers from S. cerevisiae (SEQ ID NOS 14 and 27), C albicans (SEQ ID NOS 13 and 26), L. pneumophila (SEQ ID NOS 7 and 20), M. pneumoniae (SEQ ID NOS 11 and 24), as well as genomic DNA from these four organisms. The PCR products were labeled with a fluorescent Alexa Fluor 488 dye (from Molecular Probe). The labeled PCR products were hybridized to a oligonucleotide microarray under stringent conditions. The microarray consisted of two probes each from these four organisms (i.e. SEQ ID NOS 56, 62, 65, 66, 70, 76, 79, and 80). After washing away the unhybridized PCR products, the slide was scanned in a fluorescent scanner and analyzed. All the probes hybridized, suggesting that the FtsY fragment from these four organism were amplified in a single PCR reaction using the “universal primers”. In a separate experiment, labeled PCR from each organism was hybridized to the same oligonucleotide array, under the same conditions. The result were that only the corresponding probe hybridized, suggesting that the hybridization pattern observed in the previous experiment was due to specific hybridization.

### 10. Simultaneous Identification of Bacteria and Virus

Other than approximately forty bacteria, a number of RNA viruses can also cause pneumonia. It would be beneficial to design a test that can identify these bacteria and viruses in a single assay. Since the evolution of virus is very different from that of bacteria, it is difficult to find a locus that is conserved in both bacteria and viruses. However, certain viral functions are conserved within a subgroup, such as enzyme(s) involved in replicating their genomes.

In this case, yet another embodiment of present invention is utilized. A locus or loci conserved among the virus subgroup is selected. Several PCR primer pairs based on the sequences from one of the viruses are then designed. PCR is carried out to determine which primer pair(s) is compatible with bacterial PCR and the compatible primer is selected for designing similar primers (i.e. the same amplicon) for other members of the viral group, which would also be compatible with the bacterial PCR.

The PCR for a amplicon within the gene encoding human RSV L protein (a polymerase) was found to be compatible with the PCR for bacterial RecA. Both viral and bacterial sequences were amplified in a single PCR, using a primer mixture (SEQ ID NOS 34, 47, 36, 49, 105, 106). The PCR product was fluorescently labeled with Alexa Fluor 488 (Molecular Probes) and hybridized to a microarray panel spotted with the relevant probes (SEQ ID NOS 89, 90, 101, 102, 107, 108). After stringent hybridization and wash, the slide was scanned in a fluorescent scanner. All spots showed hybridization. In a separate experiment, labeled individual PCR product from the virus or the bacteria was hybridized to the same panel and only specific hybridizations were observed.

### 11. Using Double Loci for Microorganism Identification

Given the importance of accuracy in a diagnostic test, the feasibility and usefulness of using two different conserved loci for microorganism identification were explored. It is very unlikely for two highly conserved proteins acquire sporadic mutations simultaneously. Hence, built-in redundancy helps to reduce the false-positive or negative identification caused by sequence variations. Also, using multiple probes for each pathogen helps to avoid artifacts introduced during hybridization. In a microarray format, adding extra set(s) of probes to the same panel does not add much to fixed and variable costs of the assay.

This concept was validated by using FtsY and RecA loci for the identification for twelve different bacteria: Mycoplasma pneumoniae, Chlamydia pneumoniae, Legionella pneumophila, Haemophilus influenzae, Enterococcus faecalis, Klebsiella pneumoniae, Staphylococcus aureus, Pseudomonas aeruginosa, Streptococcus pneumoniae, Bacteroides fragilis, Neisseria meningitidis, and E. coli. First, the FtsY and RecA sequences from each of the bacteria was amplified and individually labeled. Each PCR product was then hybridized to a microarray panel containing all the relevant probes under stringent conditions. For a particular pathogen, only the corresponding FtsY and RecA probes were hybridized. To confirm that there was no cross-hybridization between FtsY and RecA, RecA sequences from these pathogens were amplified and labeled in two groups, then hybridized to the same microarray panel mentioned above. Now, only RecA probes were hybridized. The same was true when FtsY sequences were amplified in two groups, and then hybridized to the same microarray.

### 12. Simultaneous Identification of Bacteria, Fungi and Viruses

In another embodiment of the invention, and to underscore the breadth of its applicability, bacteria, virus, and fungi were detected in a single assay. RNA was extracted from human RSV virus and cDNA was made using a commercial kit (Promega, Cat# A1260) SEQ ID NO 106. The cDNA was then mixed with extracted genomic DNA from Candida albicans and Mycoplasma pneumonia, and amplified through a 40-cycle PCR. The primers used were SEQ ID NOS 11, 13, 24, 26, 105, and 106. The PCR product was labeled using Alexa Fluor 546 Kit (Molecular Probes), and hybridized to a microarray panel spotted with SEQ NOS 62, 65, 76, 79, 107, and 108. All the spots hybridized, demonstrating that both the L protein locus of RSV and the FtsY loci of Candida albicans and Mycoplasma pneumonia were amplified and detected in a single assay. In a separate experiment, the specificity of the spotted probes was confirmed by hybridizing with individual PCR products.

### 13. Coupling of Oligonucleotides to Aldehyde or Epoxy Derivatized Glass Surface

One embodiment of the present invention is to carry out the hybridization in a microarray format, e.g., spotting the hybridization probes as a panel or panels onto glass surface. However, oligonucleotides does not bind to glass surface easily. Various techniques or methods have been utilized to achieve efficient coupling. In general, these method entail introducing a reactive group into the oligonucleotide and derivatizing the glass surface for coupling (Zammatteo et al, 2000). Generally, the coupling is very inefficient. This is in part due to the fact that surface reaction (where diffusion is the rate-limiting step) is less efficient than the same reaction in solution. Furthermore, part of the reaction used for coupling, i.e. Schiff's base formation, is reversible.

The present invention improves the coupling efficiency in two ways. One is to apply an electrostatic potential perpendicular to the coupling surface, drawing oligonucleotide or negatively charged molecules to the surface. The other is to use Epoxy derivatized glass surface for the coupling. The Epoxy group is preferably a three member ring, the most active one of this family of compounds. After base-catalyzed ring opening, it can react with a number of functional groups, such as —NH₂, —OH, and —SH and has been widely used to couple proteins to solid support.

For this set of experiments, an aminated oligonucleotide with a fluorescein label was used. Known amounts were spotted onto aldehyde derivatized or Epoxy derivatized glass slide, in appropriate buffer solution (50 mM carbonate buffer (pH 10.5) for Epoxy slide; 0.1M MES buffer (pH 6.5) for aldehyde slide). For charged coupling, an electric field (200 V/cm) was applied perpendicular to the slide, with the anode located underneath the slide. For coupling to aldehyde slide, the spotted slide was placed in a humidified chamber either overnight for non-charged coupling (no increase after overnight) or 48 hours for charged coupling. For coupling to Epoxy slide, the spotted slide was simply left on the benchtop for 10 minutes. At the end of the coupling reaction, the slide was washed twice with 0.1% SDS, with vigorous shaking for about two minutes each; then washed once with boiling deionized H₂O for 5 minutes. After a series of fluorescent standards were spotted onto the dried slide, the slide was scanned in a CCD-based fluorescent scanner. The percentage of coupling was defined as the amount of labeled oligonucleotide retained on the slide after the wash divided by the amount of the oligonucleotide originally spotted at the same spot. Table 3 summarizes the improvement on the coupling reaction.

TABLE 3

| Glass surface | % coupling* |
|---|---|
| Aldehyde | 0.011 |
| (non-charged) | |
| Aldehyde | 0.034 |
| (charged) | |
| Epoxy | 0.92 |

*The average of two to five data points.

Although aldehyde-derivatized glass slides are widely used for coupling aminated oligonucleotides, it is quite inefficient. Applying an electric field increases the coupling efficiency three-fold. The electric field likely moves more negatively charged oligonucleotides to the glass surface, leading to an increase in local concentration and hence more coupling. The Epoxy-derivatized glass surface generates more efficient coupling. In fact, a saturated mono-layer of oligonucleotides on the glass slide can be easily achieved with this method, though it is undesirable for subsequent hybridization reactions. It is conceivable that the coupling efficiency may be further improved by altering other parameter(s), such as increasing the voltage potential across the coupling surface or reducing the ionic strength of the oligonucleotide solution (e.g. lowering the buffer concentration).

### 14. Cloning and Sequencing of B. fragilis FtsY PCR Fragment

The FtsY gene sequence from B. fragilis has not been published prior to this invention. Based on the present invention, an equimolar primer mixture was made using FtsY primers from E. coli (SEQ ID NOS 1 and 15), Chlamydia pneumoniae (SEQ ID NOS 8 and 21), Enterococcus faecalis (SEQ ID NOS 4 and 17), Haemophilus influenzae (SEQ ID NOS 3 and 16), Legionella pneumophila (SEQ ID NOS 7 and 20), Mycoplasma pneumoniae (SEQ ID NOS 11 and 24), Staphylococcus aureus (SEQ ID NOS 10 and 23), Streptococcus pneumoniae (SEQ ID NOS 9 and 22), Neisseria meningitidis (SEQ ID NOS 6 and 19). When this primer mixture and B. fragilis genomic DNA were used in a standard PCR reaction, a single fragment about 300 bp in length was amplified. After further delineation, it was determined that SEQ ID NOS 4 and 17 were the most effective primer pair, and was in turn used as the primer pair to obtain the sequence of the amplified B. fragilis fragment by dideoxy method (SEQ ID NOS 113). For those who are skilled in the art, other methods can also be used to determine the sequence of the amplified B. fragilis PCR fragment.

### 15. Testing Cerebrospinal Fluids for Meningitis and Encephalitis

Despite advances in antiviral therapy over the past 2 decades, herpes simplex encephalitis (HSE) remains a serious illness with significant risks of morbidity and death. HSE occurs as 2 distinct entities. In children beyond the neonatal period and in adults, HSE usually is localized to the temporal and frontal lobes and is caused by herpes simplex virus type 1 (HSV-1). In neonates, however, brain involvement is more often diffuse and the usual cause is herpes simplex virus type 2 (HSV-2), which is acquired at the time of delivery. HSE is distinguished from herpes simplex meningitis, which usually is caused by HSV-2 and occurs in association with a concurrent herpetic genital infection. Like other forms of viral meningitis, it usually follows a benign course and is not discussed in this article.

Other than the herpes simplex viruses, several bacteria are also known to cause meningitis and encephalitis. It is therefore beneficial to design a test that can identify these bacteria and viruses in a single assay.

A Cluster of Orthologous Groups (COG) is a cluster of very similar proteins found in at least three species. (see Tatusov I and Tatusov II). The presence (or absence) of a protein in different genomes can tell us about the evolution of the organisms, as well as point to new drug targets. In one embodiment, the loci used for identification of bacteria and viruses were selected from COGs found in the organisms that correlate to meningitis and encephalitis. A complete list of COGs are available from the National Institutes of Health web site: http://www.ncbi.n1m.nih.gov/COG/.

Sequences of the following pathogens: Herpes Simplex Virus 1, Herpes Simplex Virus 2, Streptococcus pneumoniae, Streptococcus agalactiae (group B), Neisseria meningitidis, Hemophilus influenzae, and Listeria monocytogenes, were analyzed as follows. For each pathogen (except HSV-1 and HSV-2), an amplicon was designed for each locus and tested. For each amplicon, two capture probes were designed and tested in a microarray format. The following COGs were identified for use in the meningitis and encephalitis panel: FtsY (COG0552), 3-phosphoglycerate kinase (COG0060), and isoleucyl-tRNA synthetase (COG0126)

### 16. Designing Panels of Suitable Clusters of Orthologous Groups (COGs)

Several COGs were analyzed for specific properties other than similarity of proteins that would make them suitable for use in panels (e.g. microarrays) for testing specific pathogenic conditions. The seletion was made according to the following criteria: (i) they are highly conserved in all three branch of living organisms (i.e., bacterium, eukaryote, and archeabacterium); (ii) they are present in single-copy in most genomes; and (iii) they comprise at least two highly conserved domains flanking a more divergent domain of the coding sequence greater than 200 base pairs in length. The divergent domain may be 250, 300, 400 or 500 base pairs in length. Some examples of the loci satisfying these characteristics are listed in Table 4. All loci in Table 4 are at least as useful as (or better than) the rDNA locus. Additional loci that satisfy these criteria may become available with further analysis and sequencing and are contemplated to be included within the scope of this invention.

TABLE 4

| COG# | LOCUS |
|---|---|
| COG0173 | Aspartyl-tRNA synthetase |
| COG0525 | Valyl-tRNA synthetase |
| COG0112 | Glycine hydroxymethyltransferase |
| COG0552 | FtsY |
| COG0126 | 3-phosphoglycerate kinase |
| COG0060 | Isoleucyl-tRNA synthetase |
| COG0541 | Ffh |
| COG0215 | Cysteinyl-tRNA synthetase |
| COG0016 | Phenylalanyl-tRNA synthetase alpha subunit |
| COG0525 | Valyl-tRNA synthetase |
| COG0533 | Metal-dependent proteases |
| COG0201 | SecY |
| COG0030 | Dimethyladenosine transferase (rRNA methylation) |
| COG0193 | Peptidyl-tRNA hydrolase |
| COG0143 | Methionyl-tRNA synthetase |
| COG0173 | Aspartyl-tRNA synthetase |
| COG0124 | Histidyl-tRNA synthetase |
| COG0180 | Tryptophanyl-tRNA synthetase |
| COG0149 | Triosephosphate isomerase |
| COG0112 | Glycine hydroxymethyltransferase |
| COG0575 | CDP-diglyceride synthetase |
| COG0101 | Pseudouridylate synthase (tRNA psi55) |
| COG0064 | Glu-tRNAGln amidotransferase β subunit (PET112 homolog) |
| COG0036 | Pentose-5-phosphate-3-epimerase |

All publications and patent applications cited in this specification are hereby incorporated by reference as if each individual publication or patent application were specifically and individually indicated to be incorporated by reference in their entirety.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

Nucleotide Sequences

The following nucleotide and protein sequences are referred to and were utilized in various examples mentioned and/or described in the Specification. All of the sequences were obtained through NCBI server (publicly available database), except as otherwise noted.

| | |
|---|---|
| Fungi | |
| Candida albicans | SDSTC_5476\|C.albicans_{—} |
| | Contig5-3266 |
| Saccharomyces cerevisiae | Genbank\|M555l7.1 |
| Bacteria | |
| E. coli | Genbank\|X04398.1 |
| Bacteroides fragilis | SEQ ID NO 113 |
| Chlamydia pneumoniae | Genbank\|AE00l677.1 |
| Enterococcus faecalis | TIGR\|gef_10288 |
| Haemophilus influenzae Rd | Genbank\|U32760.1 |
| Legionella pneumophila | CUCGC_446\|lpneumo_5H86D8.1634-R |
| Kiebsiella pneumoniae | WGUSC_573\|kpneumo_B_KPN. |
| | Contig894 |
| Mycoplasma pneumoniae | Genbank\|AE000040.1 |
| Pseudomonas aeruginosa | Genbank\|AF214677.1 |
| Staphylococcus aureus | OUACGT_1280\|s.aureus_Contig474 |
| Streptococcus pneumoniae | TIGR\|S.pneumoniae_3476 |
| Neisseria meningitidis (B) | Genbank\|AE002363.1 |

| | |
|---|---|
| Bacteria | |
| E. coli | Genbank\|AE000354.1 |
| Bacteroides fragilis | Genbank\|M63029.1 |
| Chlamydia pneumoniae | Genbank\|AE001658.1 |
| Enterococcus faecalis | TIGR unfinished E.faecalis |
| | genome Contig 10288 |
| Haemophilus influenzae Rd | Genbank\|U32741.1 |
| Legionella pneumophila | Genbank\|X55453.1 |
| Klebsiella pneumoniae | WUGSC\|B_KPN.CONTIG720 |
| Mycoplasma pneumoniae | Genbank\|U00089 |
| Pseudomonas aeruginosa | Genbank\|X52261.1 |
| Staphylococcus aureus | Genbank\|L25893.1 |
| Streptococcus pneumoniae | Genbank\|Z17307.1 |
| Neisseria meningitidis | Genbank\|AE002494.1 |
| Viruses | |
| Human parainfluenza virus 1 L protein | Genbank\|AF117818.1 |
| Human parainfluenza virus 2 L protein | Genbank\|X57559.1 |
| Human parainfluenza virus 3 L protein | Genbank\|U51116.1 |
| Human respiratory syncytial virus (L) | Genbank\|U39662.1 |
| Influenza A virus, polymerase 1 | Genbank\|J02l5l.1 |
| Influenza B virus, polymerase 1 | Genbank\|NC_002204.1 |

### REFERENCES

The following patents and technical literature are relevant to this invention and may be referred to in the specification of this application and are incorporated herein by reference in their entirety.

| | |
|---|---|
| U.S. Pat. Nos. | |
| 5,989,821 | Universal Targets for Species Identification |
| 5,708,160 | HSP-60 Genomic Locus and Primers for Species |
| | Identification |
| 5,744,305 | Arrays of materials attached to a substrate. |
| 5,702,885 | Method for HLA typing |
| 5,574,145 | Isolated nucleic acid molecules targeted to the |
| | region intermidiate to the 16S and 23S rRNA genes |
| | useful as probes for determining bacteria. |
| 5,620,847 | Methods and reagents for detection of bacteria in |
| | cerebrospinal fluid. |
| 5,919,617 | Methods and reagents for detecting fungal pathogens |
| | in a biological sample. |
| 5,849,492 | Method for rapid identification of prokaryotic and |
| | eukaryotic organisms. |
| 5,187,060 | Detection of influenza a virus by polymerase chain |
| | reaction (PCR) preceded by reverse transcription of |
| | a region of the viral hemagglutinin gene |
| 5,945,282 | Hybridization probes derived from the spacer region |
| | between the 16S and 23S rRNA genes for the detection |
| | of non-viral microorganisms |
| Foreign Patents: | |
| DE19716456 | Identification of microorganisms, especially |
| | respiratory tract pathogens. |
| FR2775002 | Simultaneous detection of at least two respiratory |
| | pathogens by multiplex polymerase chain reaction |

### PUBLICATIONS

Abele-Horn. M., Busch, U., Nitschko, H., Jacobs, E., Bax, R., Pfaff, F., Schaffer, B., Heesemann, J. (1998). Molecular approaches to diagnosis of pulmonary diseases due to Mycoplasma pneumoniae. J. Clin. Microbiol 36(2): 548-551.

Bronstein, I., Voyta, J. C., Lazzari, K. G., Murphy, O., Edwards, B., Kricka, L. J. (1990). Rapid and sensitive detection of DNA in Southern blots with chemiluminescence. Biotechniques, 8(3):310-314.

Kayser, F. H. (1992). Changes in the spectrum of organisms causing respiratory tract infections: a review. Postgrad Med J. 68 (Suppl 3): S17-23.

Nielsen, P. E., Egholm, M., Berg, R. H., Buchardt, O. (1991). Sequence-selective recognition of DNA by strand displacement witha thymine-substituted polyamide. Science 254:1497-1500.

Ramirez, J. A., Ahkee, S., Tolentino, A., Miller, R. D., Summersgill, J. T. (1996). Diagnosis of Legionella pneumophila, Mycoplasma pneumoniae, or Chlamydia pneumoniae lower respiratory infection using the polymerase chain reaction on a single throat swab specimen. Diagn Microbiol Infect Dis 24(1):7-14.

Rose, T. M., Schultz, E. R., Henikoff, J. G., Pietrokovski, S., McCallum, C. M., Henikoff, S. (1998). Consensus-degenerate hybrid primers for amplification of distantly-related sequences. Nucl Acids Res; 26:1628-1635.

Saiki, R. K., Bugawan, T. L., Horn, G. T., Mullis, K. B., Erlich, H. A. (1986). Analysis of enzymatically amplified beta-globin and HLA-DQ alpha DNA with allele-specific oligonucleotide probes. Nature 324(6093):163-166.

Soomets, U, Hallbrink, M., Langel, U. (1999). Antisense properties of peptide nucleic acids. Front. Biosci. 4:D782-D786.

Southern, E. M. (1975). Detection of specific sequences among DNA fragments separated by gel electrophoresis. J Mol Biol 98(3):503-517.

Tan, J. S. (1999). Role of ‘atypical’ pneumonia pathogens in respiratory tract infections. Can. Respir. J. 6 (Supp. A): 15A-19A.

Tatusov R L, Koonin E V, Lipman D J. (1997). A genomic perspective on protein families. Science; 278(5338):631-637. (“Tatusov I”)

Tatusov R L, Natale D A, Garkavtsev I V, Tatusova T A, Shankavaram U T, Rao B S, Kiryutin B, Galperin M Y, Fedorova N D, Koonin E V. (2001) The COG database: new developments in phylogenetic classification of proteins from complete genomes. Nucleic Acids Res; 29(1):22-28. (“Tatusov II”)

Tunkel A. R. and Scheld W. M. (1993). Pathogenesis and pathophysiology of bacterial meningitis. Microbiol Rev 6(2): 118-136.

Weiler, J., Gausepohl, H., Hauser, N., Jensen, O. N., Hoheisel, J. D. (1997). Hybridisation based DNA screening on peptide nucleic acid (PNA) oligomer arrays. Nucleic Acids Res. 25(14):2792-9

Zammatteo, N., Jeanmart, L., Hamels, S., Courtois, S., Louette, P., Hevesi, L., Remacle, J. (2000) Comparison between different strategies of covalent attachment of DNA to glass surface to build DNA microarrays. Anal. Biochem., 280:143-15.

What is claimed is:

1. A method of identifying an organism among a population of organisms in a biological sample, the method comprising:
obtaining genetic material from the sample;
contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and is characteristic of the organism;
amplifying the target sequence;
contacting a solid support comprising a probe substantially complementary to the target sequence with the amplified target sequence; and
detecting hybridization of the target sequence to the probe, wherein hybridization is indicative of the presence of the organism in the sample.

2. The method of claim 1, further comprising diagnosing a disease or disorder associated with an organism, wherein hybridization of the target sequence to the probe is indicative of the presence of the organism in the sample and correlating the organism to the disease or disorder.

3. The method of claim 1, wherein the organism is selected from the group consisting of a prokaryotic organism, viral organism or a single cell eukaryotic organism.

4. The method of claim 1, wherein the prokaryotic organism is a gram positive or gram negative bacteria.

5. The method of claim 1, wherein the biological sample is a fluid sample.

6. The method of claim 5, wherein the fluid sample is blood, urine, cerebrospinal fluid, sputum, tracheal aspirate or pleural fluid.

7. The method of claim 1, wherein the biological sample is a tissue sample.

8. The method of claim 1, wherein the genetic material is DNA or RNA.

9. The method of claim 1, wherein the primer is an oligomer of DNA, RNA, or PNA.

10. The method of claim 1, wherein the target sequence is species specific.

11. The method of claim 1, wherein the target sequence is amplified by PCR.

12. The method of claim 10, wherein the probe is complementary to the species specific target sequence.

13. The method of claim 1, wherein the solid support is selected from the group consisting of nylon, glass, silicon, polymer, plastic, ceramics, metal or optical fiber.

14. The method of claim 1, wherein the solid support is a biochip.

15. The method of claim 1, wherein the detection is by measuring radioactivity, fluorescence or chemiluminescence.

16. An array of oligonucleotide probes immobilized on a solid support, the array comprising:
a plurality of probes having a sequence corresponding to a species specific polynucleotide target sequence wherein the species specific target sequence is flanked on either side by oligonucleotide sequences that are conserved across a plurality of organisms.

17. The array of claim 16, wherein the plurality of organisms are of the same family.

18. The array of claim 16, wherein the plurality of organisms are of the same genus.

19. The array of claim 16, wherein the plurality of organisms correlate with at least one common disease or disorder.

20. A kit comprising:
at least one oligonucleotide primer complementary to a conserved region of genetic material in a population of organisms; and
a solid support having attached thereto a species-specific probe capable of hybridizing to a target sequence, the target sequence flanked by the at least one primer.

21. A method for identifying at least two organisms from a population of organisms in a biological sample, comprising:
obtaining genetic material from the biological sample;
contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and each target sequence is characteristic of one of the at least two organisms;
amplifying the target sequence;
providing a solid support comprising at least two probes selected from the at least two different organisms, wherein the at least two probes comprise sequences that are substantially complementary to the target sequence in the organism from which the probe sequences were selected;
contacting the solid support with amplification products of the amplified target sequence; and
detecting hybridization of the target sequence to the probe, wherein hybridization to a probe is indicative of the presence of the corresponding organism in the sample.

22. The method of claim 21, wherein the at least two different organisms are selected from the group consisting of bacteria, yeast, paramecia, trypanosoma, unicellular eukaryotes, and viruses.

23. The method of claim 21, wherein the target sequence comprises RecA or FtsY or both.

24. A method of distinguishing a presence of at least two organisms from a population of organisms in a biological sample, comprising:
obtaining genetic material from the biological sample;
contacting the genetic material with at least a first primer and at least a related second primer corresponding to a pair of conserved regions in the genome of the population of organisms, wherein the first primer hybridizes upstream and the second primer hybridizes downstream of a target sequence in the genetic material in the sample, and further wherein the target sequence is less conserved than the primer binding sequences and each target sequence is characteristic of one of the at least two organisms;
amplifying the target sequence;
providing a solid support comprising at least two probes selected from the at least two different organisms, wherein the at least two probes comprise sequences that are substantially complementary to the target sequence and differentially hybridize to the target sequence depending on a hybridization condition;
contacting the solid support with amplification products of the amplified target sequence under a hybridization condition wherein hybridization to a probe corresponding to any one of the at least two organisms is preferred; and
detecting hybridization of the target sequence to the probe corresponding to any one of the at least two organisms, wherein hybridization to the probe is indicative of the presence of the corresponding organism in the sample.

25. The method of claim 24, wherein the hybridization condition comprises stringency or temperature or both.

26. The method of claim 24, wherein the at least two different organisms are selected from the group consisting of bacteria, yeast, paramecia, trypanosoma, unicellular eukaryotes, and viruses.

27. The method of claim 24, wherein the target sequence comprises RecA or FtsY or both.

28. A method of identifying a target sequence in a biological sample, comprising:
obtaining genetic material from the biological sample;
contacting the genetic material with at least a first primer and at least a related second primer corresponding to a pair of conserved regions in the genome of a population of organisms, wherein the first primer hybridizes upstream and the second primer hybridizes downstream of a target sequence in the genetic material in the sample, and further wherein the target sequence is less conserved than the primer binding sequences;
amplifying the target sequence; and
determining the sequence of amplification products of the amplified target sequence.

29. The method of claim 28, further comprising:
identifying an organism associated with the sequenced target sequence by comparing the sequence of the amplified target with a known sequence of the corresponding target in the organism.

30. The method of claim 29, wherein the organism comprises a bacteria, a yeast, an unicellular eukaryote or a virus.

31. The method of claim 29, wherein the target sequence comprises RecA or FtsY or both.

32. A probe corresponding to a RecA gene wherein the probe is selected from the group consisting of polynucleotides having SEQ ID NOS 53-80.

33. A probe corresponding to a FtsY gene wherein the probe is selected from the group consisting of polynucleotides having SEQ ID NOS 81-104.

34. A probe corresponding to a human RSV virus wherein the probe is selected from the group consisting of polynucleotides having SEQ ID NOS 107 and 108.

35. A primer oligonucleotide for use as a forward PCR primer for an amplification of FtsY sequences in an organism, wherein the oligonucleotide is selected from the group consisting of:
(a) an oligonucleotide wherein five nucleotides at a 3′ end of the oligonucleotide bears at least about 80% sequence identity to five nucleotides at a 3′ end of oligonucleotides selected from the group consisting of SEQ ID NOS 1-14; and
(b) an oligonucleotide wherein the oligonucleotide bears at least about 70% sequence identity to oligonucleotides selected from the group consisting of SEQ ID NOS 1-14.

36. A primer oligonucleotide for use as a reverse PCR primer for use with a related primer of claim 35, for an amplification of FtsY sequences in an organism, wherein the oligonucleotide is selected from the group consisting of:
(a) an oligonucleotide wherein wherein five nucleotides at a 3′ end of the oligonucleotide bears at least about 80% sequence identity to five nucleotides at a 3′ end of the oligonucleotides selected from the group consisting of SEQ ID NOS 15-27; and
(b) an oligonucleotide wherein the oligonucleotide bears at least about 70% sequence identity to oligonucleotides selected from the group consisting of SEQ ID NOS 15-27.

37. A primer oligonucleotide for use as a forward PCR primer for an amplification of RecA sequences in an organism, wherein the oligonucleotide is selected from the group consisting of:
(a) an oligonucleotide wherein five nucleotides at a 3′ end of the oligonucleotide bears at least about 80% sequence identity to five nucleotides at a 3′ end of oligonucleotides selected from the group consisting of SEQ ID NOS 28-40; and
(b) an oligonucleotide wherein the oligonucleotide bears at least about 70% sequence identity to oligonucleotides selected from the group consisting of SEQ ID NOS 28-40.

38. A primer oligonucleotide for use as a reverse PCR primer for use with a related primer of claim 37, for an amplification of RecA sequences in an organism, wherein the oligonucleotide is selected from the group consisting of:
(a) an oligonucleotide wherein five nucleotides at a 3′ end of the oligonucleotide bears at least about 80% sequence identity to five nucleotides at a 3′ end of oligonucleotides selected from the group consisting of SEQ ID NOS 41-52; and
(b) an oligonucleotide wherein the oligonucleotide bears at least about 70% sequence identity to oligonucleotides selected from the group consisting of SEQ ID NOS 41-52.

39. A primer oligonucleotide for use as a forward PCR primer for an amplification of a human RSV sequence, wherein the oligonucleotide is selected from the group consisting of:
(a) an oligonucleotide wherein five nucleotides at a 3′ end of the oligonucleotide bears at least about 80% sequence identity to five nucleotides at a 3′ end of oligonucleotides selected from the group consisting of SEQ ID NO 105; and
(b) an oligonucleotide wherein the oligonucleotide bears at least about 70% sequence identity to oligonucleotides selected from the group consisting of SEQ ID NO 105.

40. A primer oligonucleotide for use as a reverse PCR primer for use with a related primer of claim 39, for an amplification of a human RSV sequence, wherein the oligonucleotide is selected from the group consisting of:
(a) an oligonucleotide wherein five nucleotides at a 3′ end of the oligonucleotide bears at least about 80% sequence identity to five nucleotides at a 3′ end of oligonucleotides selected from the group consisting of SEQ ID NO 106; and
(b) an oligonucleotide wherein the oligonucleotide bears at least about 70% sequence identity to oligonucleotides selected from the group consisting of SEQ ID NO 106.

41. A polynucleotide comprising SEQ ID NO 109, wherein the polynucleotide corresponds to a FtsY gene of Bacteroides fragilis.

42. A hybridization probe for use in a detection of a FtsY gene comprising a selectively hybridizable segment of the polynucleotide of claim 41.

43. A PCR primer oligonucleotide sequence for use in detection of a FtsY gene, wherein the PCR primer oligonucleotide sequence comprises at least five continuous nucleotides of a polynucleotide comprising SEQ ID NO 109.

44. A method for increasing the efficiency of coupling of an oligonucleotide to a solid substrate, the method comprising:
applying a positive electrostatic potential to a surface of the solid substrate, whereby the positive electrostatic potential increases a concentration of oligonucleotides and negatively charged molecules to the surface of the solid substrate.

45. A method for increasing the efficiency of coupling of an oligonucleotide to a glass substrate by forming an Epoxy derivative of a surface of the glass substrate, the method comprising:
applying an epoxy derivative to the surface of the glass substrate.

46. The method of claim 45, wherein the epoxy comprises a three member ring.

47. An array for testing a biological fluid for the presence of at least one organism correlated with at least one condition selected from meningitis and encephalitis, the array comprising:
at least two probes comprising sequences of clusters of orthologous groups (COGs) selected from at least one of FtsY (COG0552), 3-phosphoglycerate kinase (COG0060), and isoleucyl-tRNA synthetase (COG0126).

48. The array of claim 47 wherein the organism is selected from the group consisting of Herpes Simplex Virus 1, Herpes Simplex Virus 2, Streptococcus pneumoniae, Streptococcus agalactiae (group B), Neisseria meningitidis, Hemophilus influenzae, and Listeria monocytogenes.

49. A method for selecting one or more COGs for use in an array for detecting at least one organism related to a pathologic condition, the method comprising:
examining a sequence of at least one COG from an organism related to the pathologic condition; and
selecting one or more COGs wherein the selected COGs satisfy a criteria comprising
(i) the COG is highly conserved in bacterium, eukaryote, and archeabacterium;
(ii) the COG is present in a single-copy in a genome; and
(iii) the COG comprises at least two highly conserved domains flanking a more divergent domain of the coding sequence greater than 200 base pairs in length.

50. The method of claim 49 wherein the COG is selected from the group consisting of COG0173 (aspartyl-tRNA synthetase), COG0525 (valyl-tRNA synthetase), and COG0112 (glycine hydroxymethyl transferase).

51. The method of claim 49 wherein the COG is selected from a COG listed in Table 4.
